(19) United States
(12) Patent Application Publication
(10) Pub. No.: US 2026/0165425 A1
(43) Pub. Date: Jun. 18, 2026

(54) SYSTEMS, METHODS AND APPARATUS FOR PROVIDING PERSONALIZED FOOTWEAR

(71) Applicant: Iambic Inc., New York, NY (US)

(72) Inventors: Maeve T. Wang, New York, NY (US); Chaudhry Raza Hassan, Rego Park, NY (US); Morteza Haeri-Hosseini, Gainesville, FL (US); Tunyanun Deselm, South Bend, IN (US)

(21) Appl. No.: 18/844,568

(22) PCT Filed: Mar. 8, 2023
(86) PCT No.: PCT/US2023/014772

§ 371 Date: Sep. 6, 2024

Related U.S. Application Data
(60) Provisional application No. 63/318,272, filed on Mar. 9, 2022.

(51) Int. Cl. A43B 23/02; A61B 5/103

(52) U.S. Cl. CPC A43B 23/0205; A61B 5/1036

(57) ABSTRACT

In some embodiments, systems, apparatuses and methods are provided herein useful to providing personalized footwear, including designing and providing personalized modular lasts and personalized shoe components. In one aspect, a design for a personalized modular last is generated from assessment information associated with a user, the assessment information comprising at least one foot measurement of the user, and optionally comprising other foot shape information, pathology information, gait and biomechanics information, or contextual information associated with the user. Generating the design for the personalized modular last includes determining a modular core last model by selecting one or more modular core last model components from a library of modular core last model components, based on the assessment information associated with the user. Last extensions may further be determined to further personalize the design for the personalized modular last. In a further aspect, a design for at least one personalized shoe component is generated based on the design for the personalized modular last and/or the assessment information associated with the user. Methods of manufacturing the personalized footwear are also provided, as well as using previously worn footwear feedback to update a personalized footwear design.

### RELATED APPLICATIONS

This application claims the benefit of U.S. Provisional Application No. 63/318,272, filed Mar. 9, 2022, which is incorporated herein by reference in its entirety.

### TECHNICAL FIELD

This invention relates generally to personalized footwear, and more specifically, to designing and manufacturing personalized footwear based on an assessment of an individual's footwear needs.

### BACKGROUND

Footwear fit has potential impacts on a human, industry, and global level. Poor footwear fit may cause personal pain to a wearer and has deep implications across the entire footwear industry value chain—from footwear design to manufacturing to retail. At a human level, foot health may play a central role in mobility and quality of life. Studies have shown that 63% to 72% of people wear ill-fitting footwear, the impact of which extends far beyond personal pain, resulting in deformities and disorders. Indeed, improperly fitting footwear has been shown to be associated with a number of medically related disorders (e.g., reduced short- and long-term mobility, lower quality of life, etc.) Discovering new, better-fitting footwear has generally been a manual trial-and-error process that wastes a consumer's time and money, and results in footwear that does not fit or footwear that causes pain.

The footwear industry's current sizing system oversimplifies fit and fails to capture the broad spectrum of foot morphologies, pathologies, and gait and biomechanics. For example, individuals with medical conditions (e.g., digital deformities, arthritis, diabetes, etc.) and older adults that have geriatric needs are hard to address through any existing footwear sizing/selection systems.

Today, the footwear industry is poorly equipped to enable proper fit. For one, there is no industry standard for footwear sizing, which may vary significantly across brands, footwear types, models, and annual versions, at a minimum. This makes it very difficult for the customer to locate a correct fit. Further, the industry's standard approach to sizing is also two-dimensional, based on foot width and length. This is underscored by the industry's typical use of shoe lasts, which are mechanical forms, often made of wood, metal, or plastic, that are shaped like a human foot and used to set the size, silhouette, and shape of the shoe during construction. While shoe lasts will vary based on the type of shoe (e.g., a man's boot, a woman's oxford), shoe lasts for a given type or model of shoe have sizes and dimensions which change or “grade” in a systematic way, parallel to shoe sizing. This limited sizing of shoe lasts is compounded by the fact that shoe lasts tend to be symmetrical, the right side a mirror image of the left. The human anatomy fails to obey such rules of size and symmetry. As such, the industry fails to account for the foot's intricate shape, with its 26 bones, 33 joints, 100+ligaments, tendons, and muscles. Nor does it account for other factors, such as personal gait: footwear are devices that enable people to move, devices that interact with each foot's 7,000+nerve endings which send signals to the rest of the body in motion. Unlike garment fit, which can be approximate, footwear fit that is imperfect—without considering the foot's complexities in shape and movement—causes pain, a physiological sign of biofeedback that something is harmful to the body. Given the complex interaction between footwear and the human body's anatomy and physiological traits, understanding the science of footwear fit is critical to making appropriate footwear recommendations and resolving fit-induced returns.

Moreover, part of the issue also lies upstream in the footwear industry's value chain. Footwear design practices remain reliant on basic trial- and error with qualitative feedback from a limited sample of foot models. Many footwear manufacturers lack the necessary data to optimize the designs of mass manufactured shoes to fit broader segments of the population. Even if footwear manufactures are considering personalizing footwear for consumers, mass manufacturing techniques today are too inefficient to do so, resulting in wasted resources and labor. For this reason, custom-made footwear remains handcrafted, a costly and unscalable process, which drives the high cost and thereby price point for custom shoes beyond the reach of average consumers.

At present, customization or personalization of shoes can include creating custom shoe components (such as custom insoles or padding) and/or focusing on the shoe last. For example, in one approach to making customized shoes, a pre-manufactured last for creating shoes may be chosen based on the length and width of a customer's foot. In this approach, the details and materials of the shoe components may be customized to meet some of the customer's needs, manufactured by one of the above-mentioned manufacturing techniques. However, since the actual size and shape of the shoe is based on the pre-manufactured last, which merely approximates the customer's foot, the customer's individual shape differences, such as the height of arch or the shape of a heel, are not fully captured by this approach.

In another approach, a shoemaker may build customized lasts for each individual based on the sizing of that individual's feet, but this can be a wasteful, time-consuming process that lacks scalability, and is typically very costly for the customer. Further, this approach also typically fails to integrate a wide range of biometric, geometric, and contextual data relevant to achieving a user's ideal fit.

### BRIEF DESCRIPTION OF THE DRAWINGS

Disclosed herein are embodiments of systems, apparatuses and methods pertaining to providing personalized footwear. This description includes drawings, wherein:

FIG. 1 is a flow diagram of a method for providing personalized footwear, in accordance with some embodiments.

FIG. 2 is a flow diagram of a method for designing a personalized modular last for providing personalized footwear, in accordance with some embodiments.

FIG. 3 is a schematic illustration of an exemplary system for providing personalized footwear in accordance with some embodiments.

FIG. 4 is a flow diagram of a method for providing personalized footwear, in accordance with some embodiments.

FIG. 5 is a flow diagram of a method of analyzing image data to determine foot shape attributes associated with a user, in accordance with some embodiments.

FIG. 6 is a flow diagram of a method of acquiring and displaying foot pathology data, in accordance with some embodiments.

FIG. 7 is a flow diagram of a method of analyzing image data to determine foot pathology attributes associated with a user, in accordance with some embodiments.

FIG. 8 is a flow diagram of a method of performing a gait assessment, in accordance with some embodiments.

FIG. 9 is a flow diagram of a method of analyzing foot plantar pressure data to determine biomechanics attributes associated with a user, in accordance with some embodiments.

FIG. 10 is a flow diagram of a method of analyzing contextual data, in accordance with some embodiments.

FIG. 11 is a simplified schematic diagram of a method of designing personalized footwear to provide personalized footwear, in accordance with some embodiments.

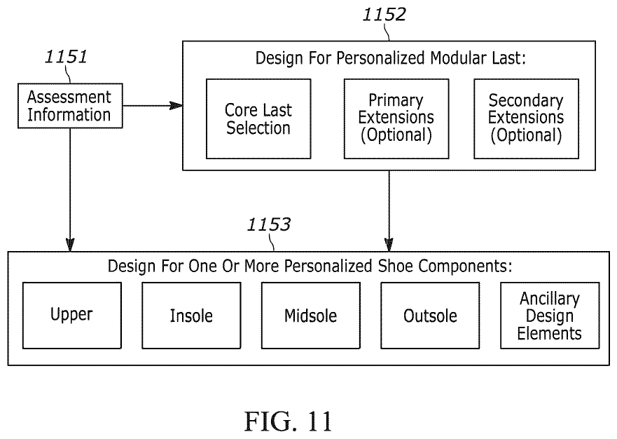

FIG. 12 is a flow diagram of a method of providing footwear data, in accordance with some embodiments.

FIG. 13 is a perspective view of a personalized modular last, in accordance with some embodiments.

FIG. 14 includes diagrammatic side views of different modular core lasts, in accordance with some embodiments.

FIG. 15 is a top plan view of a modular core last, in accordance with some embodiments.

FIG. 16 shows top plan views of regions of a personalized modular last, in accordance with some embodiments.

FIG. 17 shows views of additional regions of a personalized modular last, in accordance with some embodiments.

FIG. 18 shows a simplified schematic diagram of a method of providing a personalized shoe component design, in accordance with some embodiments.

FIG. 19 is a flow diagram of a method of providing or updating a design for personalized footwear to provide personalized footwear, in accordance with some embodiments.

FIG. 20 is a flow diagram of a method of providing or updating a design for personalized footwear to provide personalized footwear, in accordance with some embodiments.

FIG. 21 is a flow diagram of a method of analyzing sensor data to determine personal sensed attributes associated with a user, in accordance with some embodiments.

FIG. 22 is a flow diagram of a method of creating a design or digital drawing of a personalized footwear, in accordance with some embodiments.

FIG. 23 is a flow diagram of a method of updating a personal foot model, in accordance with some embodiments.

FIG. 24 is a flow diagram of a method of manufacturing and updating a design for a personalized footwear product, in accordance with some embodiments.

FIG. 25 is a flow diagram of a method of manufacturing and updating a design for a personalized footwear product, in accordance with some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to various embodiments, systems, apparatuses and methods are described herein which may be used to provide personalized footwear. More specifically, the present disclosure includes providing personalized modular lasts and personalized shoe components based on information about a user. Also described are systems and methods for providing or manufacturing personalized footwear by designing personalized modular lasts as well as personalized shoe components, based on an assessment of an individual's footwear needs. Furthermore, the teachings herein may be used in conjunction with other personalized footwear manufacturing tools, systems, and processes such as those systems, apparatuses, and methods described in U.S. Patent Application No. 63/155,171, filed Mar. 1, 2021, U.S. Patent Application No. 63/277,818, filed Nov. 10, 2021, and International Application No. PCT/US2022/017641, filed Feb. 24, 2022, all of which are incorporated herein by reference in their entireties.

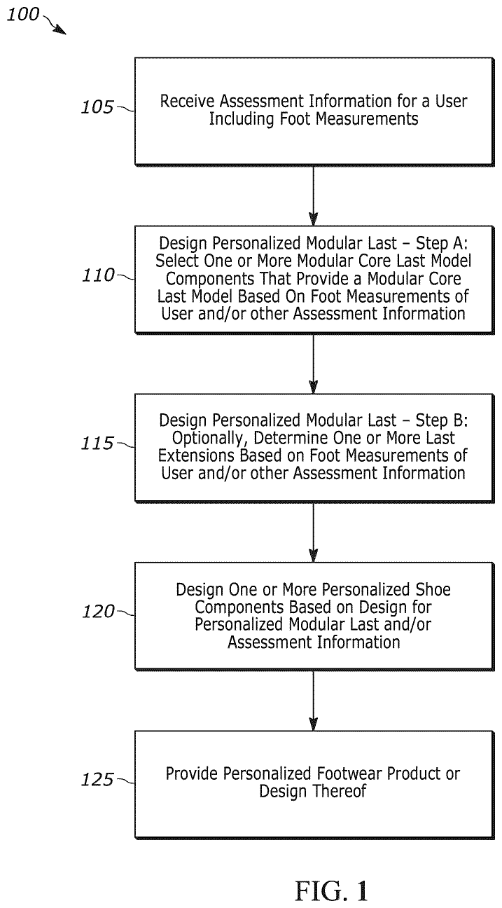

In one aspect, shown in FIG. 1, a method is disclosed for providing a personalized footwear product that includes obtaining assessment information associated with an individual or user, the assessment information comprising at least one foot measurement of the individual; generating a design for a personalized modular last, wherein the personalized modular last comprises a modular core last. In some approaches, generating the design for the personalized modular last includes determining a modular core last model by selecting one or more modular core last model components from a library of modular core last model components, based at least in part on the at least one foot measurement of the individual, or on the assessment information associated with the individual; and generating a design for at least one personalized shoe component, based at least in part on the design for the personalized modular last and/or the assessment information associated with the individual.

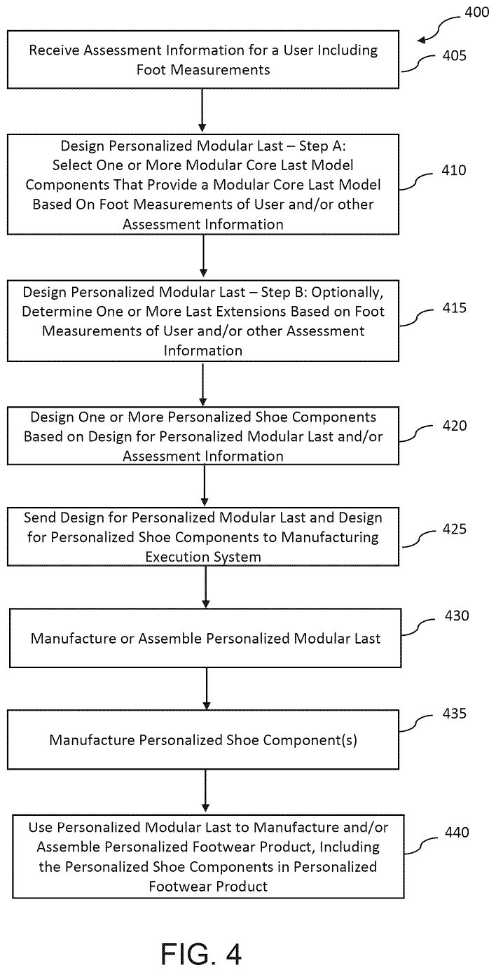

In some embodiments, the method further includes sending the design for the personalized modular last and the design for the at least one personalized shoe component to be manufactured. In some embodiments they are sent to a manufacturing execution system. Manufacturing a personalized footwear product includes providing the personalized modular last, using the personalized modular last to manufacture the personalized footwear product, and incorporating the at least one personalized shoe component into the personalized footwear product, as shown in FIG. 4.

The present disclosure further contemplates a personalized modular last for providing personalized footwear to an individual that includes a modular core last and one or more last extensions. In some approaches, the modular core last comprises one or more modular core last components selected from a group of different modular core last components based at least in part on foot measurements associated with the individual and/or the assessment information associated with the user. In some embodiments, the one or more last extensions are removably coupled to the modular core last at selected attachment points on the modular core last, and the one or more last extensions and selected attachment points may be selected based at least in part on foot measurements associated with the user and/or other assessment information associated with the user.

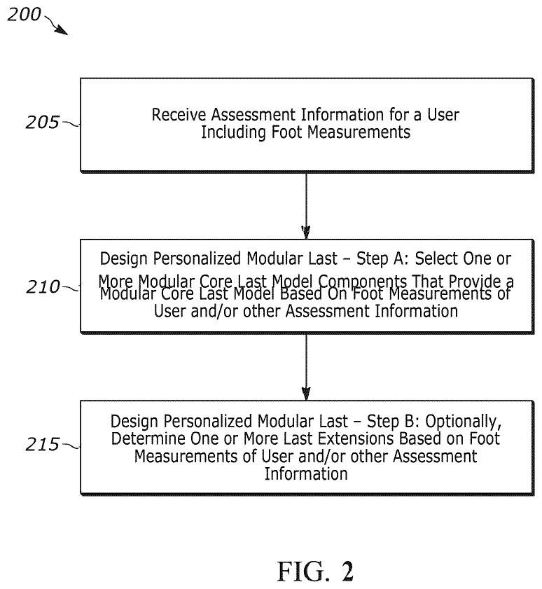

Further disclosed is a method for designing or providing a personalized modular last, as shown in FIG. 2, including the steps of receiving assessment information for a user including foot measurements, generating a design for a personalized modular last, wherein the personalized modular last comprises a modular core last. In some approaches, generating the design for the personalized modular last includes determining a modular core last model by selecting one or more modular core last model components from a library of modular core last model components, based at least in part on the foot measurements of the user and/or the assessment information associated with the user, and, optionally, determining if one or more last extensions should be included in the personalized modular last based on the assessment information associated with the user, and/or determining one or more last extensions based on the assessment information associated with the user. In some embodiments, the method may include sending the design for the personalized modular last to be manufactured or to a manufacturing execution system. In some embodiments the method includes manufacturing or otherwise providing the one or more last extensions if included, and removably coupling them to the modular core last to provide the personalized modular last. In further embodiments, the personalized modular last may be used to manufacture a personalized footwear product for the user. A corresponding system for designing or providing a personalized modular last is also disclosed.

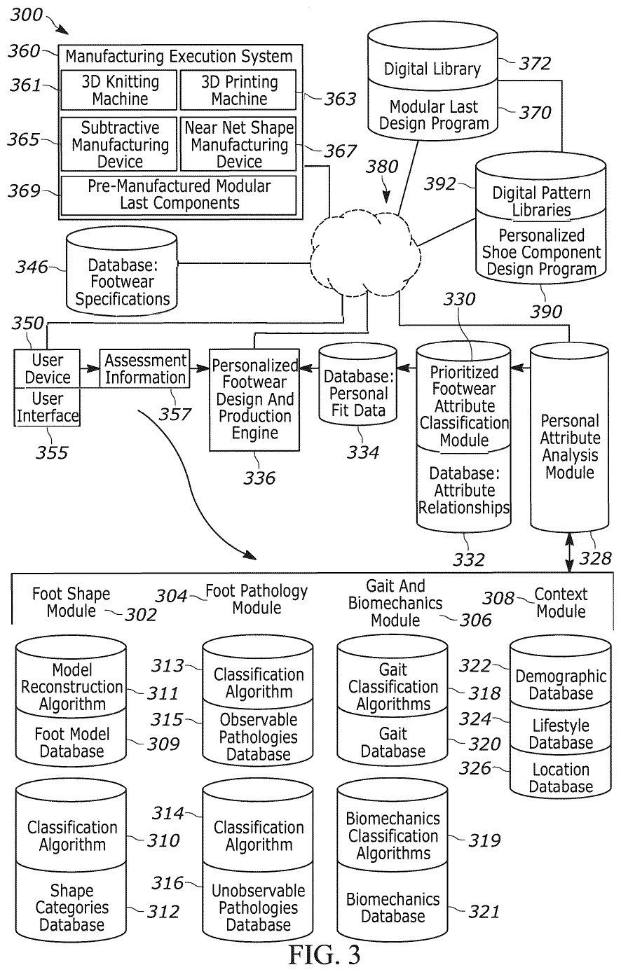

The present disclosure also contemplates a system for providing a personalized footwear product, as shown in FIG. 3, the system including at least one processor communicable with at least one electronic user device, the processor configured to receive assessment information associated with a user, the assessment information including at least one foot measurement of the user, and generate a design for a personalized modular last, wherein the personalized modular last comprises a modular core last. In some approaches, generating the design for the personalized modular last includes determining a modular core last model by selecting one or more modular core last model components from a library of modular core last model components, based at least in part on the foot measurements of the user and/or the assessment information associated with the user. The processor may also be further configured to generate a design for at least one personalized shoe component, based at least in part on the design for the personalized modular last and/or the assessment information associated with the user.

In some embodiments, the system further comprises a manufacturing execution system, wherein the processor is configured to send the design for the personalized modular last and the design for the at least one personalized shoe component to the manufacturing execution system. In such a configuration, the manufacturing execution system may be configured to provide or manufacture the at least one personalized shoe component, provide the personalized modular last, use the personalized modular last to manufacture the personalized footwear product, and include the at least one personalized shoe component in the personalized footwear product.

In another aspect, the systems and methods provided herein may provide for designing a personalized modular last and/or personalized shoe components based on comprehensive personal data associated with a user. Such personal data may include data related to the foot shape, foot pathologies, gait and biomechanics, demographics, lifestyle, location, and/or product preferences associated with a user, as well as feedback regarding a previously worn shoe.

Further disclosed is a system for providing a personalized footwear product, the system including at least one processor communicable with at least one electronic user device, the processor configured to: obtain assessment information associated with a user, where the assessment information includes at least one foot measurement of the user and generate a design for a personalized modular last, where the personalized modular last may include a modular core last. In some approaches, generating the design for the personalized modular last includes determining a modular core last model by selecting one or more modular core last model components from a library of modular core last model components. In operation, these components may be chosen from the library of components based at least in part on the at least one foot measurement of the user. Further, the processor also may be configured to generate a design for at least one personalized shoe component, based on the design for the personalized modular last and, optionally, the assessment information associated with the user.

In some approaches, the processor also is configured to obtain feedback regarding a previously worn footwear product that was manufactured based on the design for the personalized modular last and the design for the personalized shoe component. In use, the processor may be configured to generate an updated design for the personalized modular last and/or an updated design for the at least one personalized shoe component based at least in part on the feedback regarding a previously worn footwear product.

Also contemplated are methods and systems for designing one or more personalized shoe components, including obtaining assessment information associated with a user (where the assessment information may include one or more foot measurements) and generating a design for one or more personalized shoe components. In some configurations, generating the design for the one or more personalized shoe components includes, for example, using an algorithm to select a preset shoe component design from a library of preset shoe component designs based on a design for a personalized modular last personalized to the user and, optionally, the assessment information associated with the user. This approach may be leveraged for at least one or all of the personalized shoe components. In addition, the methods and systems described herein may use an algorithm to resize the selected preset shoe component design based on the design for the personalized modular last personalized to the user and, optionally, the assessment information associated with the user. Further, in some approaches, the methods and systems permit the user or design to select a material for the personalized shoe component based on the design for the personalized modular last and/or the assessment information associated with the user.

While the personalized footwear described herein may incorporate many personalized components that are generated and crafted with a particular user in mind, such footwear also typically include one or more standardized components, such as, for example, shoe laces and rivets, among others. The teachings herein allow the user or designer to identify which of the footwear components are appropriately customized and which standardized items or components may be used. Furthermore, these teachings may be employed to adjust some of the standard, off-the-shelf components to create personalized shoe components, thereby manufacturing personalized footwear in both an economical and efficient manner.

In some embodiments, the systems and methods may include determining personal attributes and/or footwear attributes associated with a user based on the user's received assessment information, and designing the personalized footwear product based at least in part on the determined personal attributes and/or footwear attributes associated with the user.

It is contemplated that the systems, apparatuses, and methods described herein may be employed by a number of potential users, including but not limited to, shoppers, consumers, businesses such as shoe retailers or shoe designers, electronic marketplaces, footwear manufacturers, footwear providers or suppliers, medical professionals, sales professionals, or any other entities involved in the provision, design, or manufacturing of footwear or footwear related products.

The following description is not to be taken in a limiting sense, but is made merely for the purpose of describing the general principles of exemplary embodiments. Reference throughout this specification to “one embodiment,” “an embodiment,” “some embodiments”, “an implementation”, “some implementations”, “some applications”, or similar language means that a particular feature, structure, or characteristic described in connection with the embodiment may be included in an embodiment. Thus, appearances of the phrases “in one embodiment,” “in an embodiment,” “in some embodiments”, “in some implementations”, and similar language throughout this specification may, but do not necessarily, all refer to the same embodiment.

As mentioned above, FIGS. 1 through 4 each illustrate an exemplary system or method in accordance with certain embodiments of the present disclosure for providing a personalized modular last or personalized footwear. As shown, assessment information about a user, at least including the foot measurements of a user, is initially received.

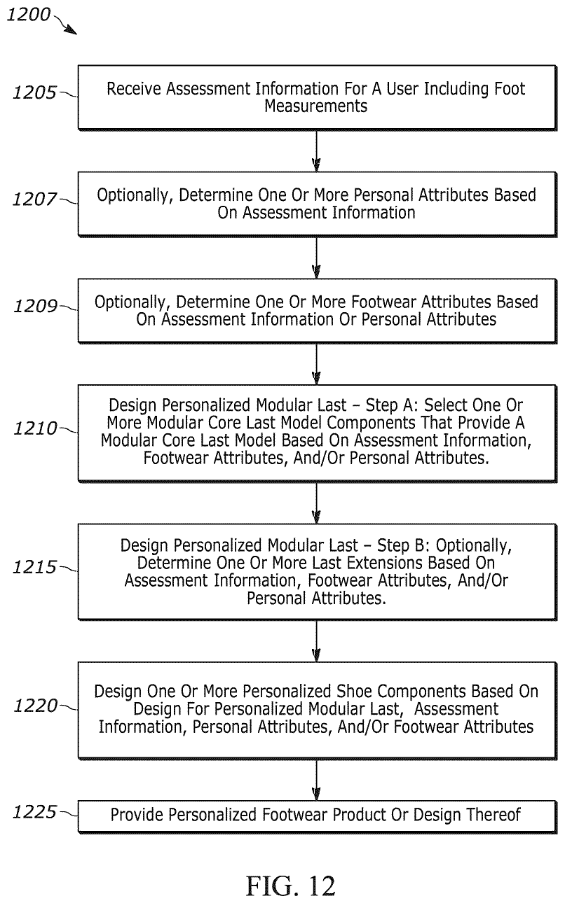

The assessment information about a user described herein includes personal data that may be received by the processor from one or more electronic user devices. For example, a user interface on an application on mobile phone or a computer may be used to provide a user's foot measurements to the processor. In some embodiments, the assessment information is received and used to design and provide the personalized modular last and/or personalized shoe components. For example, foot measurements of a user may be received and directly used to select or determine the components for the personalized last or personalized shoe components. In an alternative embodiment, shown in FIG. 12, the assessment information may be received 1205 and additionally analyzed to determine 1207, 1209 one or more personal attributes or footwear attributes associated with the user that may impact the footwear needs or calculated footwear fit for the user. The designs for the personalized modular last and, optionally, one or more personalized shoe components, may then be based at least in part on one or more of determined personal attributes or footwear attributes.

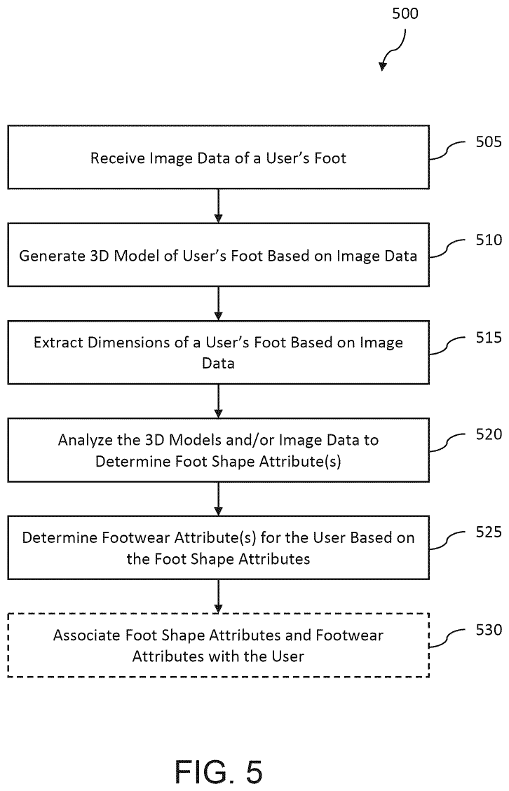

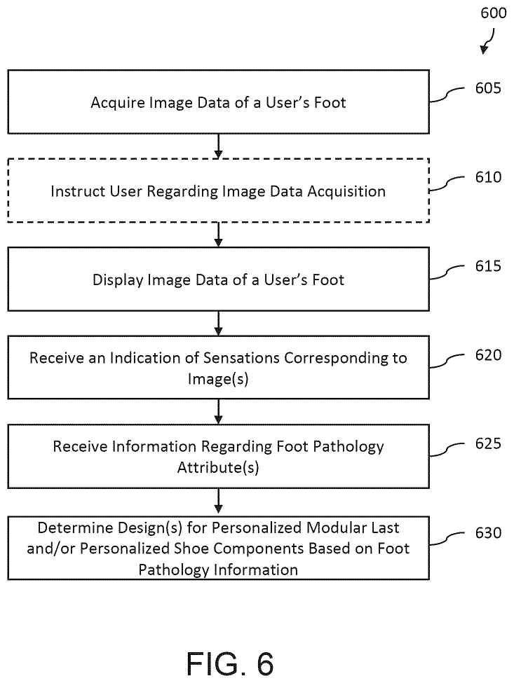

The assessment information may include various forms of personal data, such as image data associated with a user, video data associated with a user, responses to electronic questionnaires, or responses to electronic requests for information from a user. Personal data may include one or more of personal foot shape data, such as foot measurements, geometry, or morphology, personal pathology data, personal gait data, personal biomechanics data, and personal contextual data. The personal data may be received or inputted into the system by any means. In some approaches, the personal data may be collected by instructing a user, such as in the method described with reference to FIG. 6. The method may further include analyzing the personal data to determine one or more personal attributes associated with a user. Such personal attributes may include one or more qualities, characteristics, or preferences that may impact footwear needs and fit. In some embodiments, personal data may be analyzed to determine one or more personal foot shape attributes associated with a user or individual. In some embodiments, personal data may be analyzed to determine one or more personal pathology attributes associated with a user. In some embodiments, personal data may be analyzed to determine one or more gait or biomechanics attributes associated with a user. In some embodiments, personal data may be analyzed to determine one or more personal contextual attributes associated with a user. In some approaches, the personal data may be analyzed using one or more of the methods described with reference to, for example, FIGS. 5, 7, 8, 9, and 10.

In some embodiments, the method may also include determining one or more footwear attributes associated with the user based on the personal attributes or based on the assessment information associated with the user, as shown in FIG. 12. In some approaches, personal data may be analyzed via one or more modules associated with a system for providing personalized footwear, such as illustrated in the system 300 described with reference to FIG. 3.

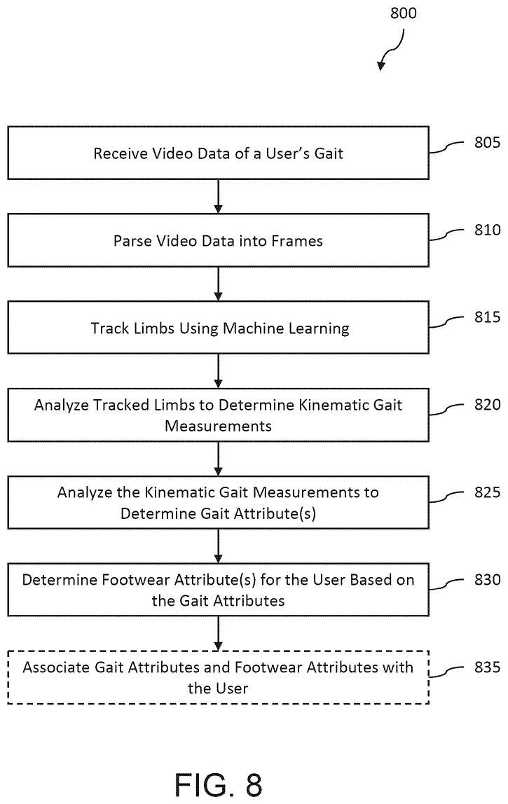

In some embodiments, gait data may be collected and used to provide the personalized modular last and/or the personalized shoe components. In one approach, the processor may determine one or more personal gait or biomechanics attributes that may impact the footwear needs and/or calculated footwear fit for a user. Such gait and biomechanics data may include various forms of data, such as image data or video data, associated with a user. For example, gait data may include a video capturing a user's gait (i.e., a user walking) under specified conditions and biomechanics data may include a pressure map of the foot plantar pressure during standing, walking, and/or doing any other activity. In some approaches, the gait and biomechanics data may be analyzed using the method described with reference to FIG. 8. The method may include tracking the user's limbs on the video and obtaining kinematic measurements by analyzing the tracked limbs. The gait data, tracked points, and kinematic measurements may be analyzed to determine personal gait information, and/or one or more gait attributes associated with the user. For example, such gait information or gait attributes may include step time, step length, gait speed, stance time, swing time, foot position, foot plantar flexion, ankle plantar flexion, knee flexion, joint loading foot loading, other lower limb kinetics, or other characterizations of the gait of the user.

An embodiment may also include determining 830 one or more footwear attributes associated with the user based on the personal gait or biomechanics information or attributes associated with the user. In some approaches, gait and biomechanics data may be analyzed to determine personalized footwear attributes via one or more modules associated with a system for providing personalized footwear, such as the system 300 described with reference to FIG. 3.

FIG. 3 illustrates an exemplary system 300 for providing personalized footwear in accordance with some embodiments. An exemplary data structure is illustrated in FIG. 3; however, it should be understood that other suitable data structures may be used by the system 300.

The components of system 300 may communicate directly or indirectly, such as over one or more distributed communication networks, such as network 380. For example, network 380 may include LAN, WAN, Internet, cellular, Wi-Fi, Bluetooth, and other such communication networks or combinations of two or more such networks. Various components of system 300 may also be hardwired.

It is contemplated that one or more processors may be associated with any of the components described in system 300. The term processor refers broadly to any microcontroller, computer, control-circuit, or processor-based device with processor, memory, and programmable input/output peripherals, which is generally designed to govern the operation of other components and devices. It is further understood to include common accompanying accessory devices, including memory, transceivers for communication with other components and devices, etc. These architectural options are well known and understood in the art and require no further description here. The processor may be configured (for example, by using corresponding programming stored in a memory as will be well understood by those skilled in the art) to carry out one or more of the methods, steps, actions, and/or functions described herein.

The system 300 may simply receive assessment information from an electronic user device in various manners, or may additionally include one or more electronic user devices 350. The user devices may be configured to receive recommendations, prompts, queries, surveys, notifications, alerts, instructions, user input, or other information. The electronic user device may include, for example, a smart phone, a tablet, a laptop, a personal computer, a smart watch, etc.

One or more user interfaces 355 may be associated with the electronic user devices 350. The user interfaces may be used for user input and/or for output display. For example, the user interface may include any known input devices, such as one or more buttons, knobs, selectors, switches, keys, touch input surfaces, audio input, and/or displays, etc. The user interfaces 355 may further include lights, visual indicators, display screens, etc. to convey information to a user, such as but not limited to the communication of: footwear recommendations; instructions regarding capturing images or video of a user's foot or gait; questions or requests for information regarding pathologies or contextual information associated with a user; prompts or instructions to indicate sensations associated with image of a user's foot; a sensation map illustrating sensations associated with an image of foot; notifications; prompts; errors, and/or other such information. Sensation maps may be either two dimensional or three dimensional. In this manner, the system may receive data or information regarding the foot shape, pathologies, gait, biomechanics, or contextual attributes associated with a user via a user interface.

The electronic user device may also be equipped with one or more sensors. The sensors may include one or more of an image sensor (e.g., a camera), a temperature sensor, a gyroscope, a light sensor, and a global positioning system (GPS) sensor. In some approaches, the image sensor may be able to record video at a frame rate of at least about 60 frames per second (fps), about 59.94 fps, about 50 fps, about 30 fps, or, in some aspects, about 24 fps. In some approaches, the image sensor may be able to record at a resolution of at least about 1440p, about 1080p, or, in some aspects, about 720p. Using an image sensor, such as a camera in an electronic user device, a user may capture one or more still images or pictures and/or one or more videos of the user's foot, limb, or portion thereof.

In one embodiment, the user may use one or more electronic user devices to complete a virtual footwear assessment by submitting at least one of the following: answers to a series of questions, one or more captured pictures of the user's feet, and one or more captured videos of the user's gait. In this manner, information may be provided to determine a personalized modular last and/or personalized shoe components to make personalized footwear with a suitable fit for the user. In one embodiment, one or more sensors and/or one or more cameras from the electronic user device may be utilized to acquire any necessary data (e.g., pictures, videos, etc.) from the user to design and provide the personalized footwear or otherwise assess the footwear needs of a user.

The system 300 may also include various modules to determine and/or analyze information used to provide personalized footwear for a user. In some embodiments, the system 300 may include one or more of a foot shape module 302, a pathology module 304, a gait and biomechanics module 306, and a context module 308. These modules may be employed by system 300 to acquire raw data and to determine, assess, or otherwise extract personal attributes associated with a user based on the raw data. Personal attributes (e.g., foot shape attributes, pathology attributes, gait and biomechanics attributes, contextual attributes) may include a collection of an individual's personal qualities, characteristics, and product preferences that may influence calculated footwear fit and footwear personalization. For example, personal attributes may include: foot shape attributes such as 3D foot shape, 3D foot dimensions, 2D foot shape, and 2D foot dimensions; foot pathology attributes such as pains, conditions, unobservable pathologies, observable pathologies, and other foot-related medical conditions; gait attributes such as gait patterns, measurements, and kinematics; biomechanics attributes such as foot plantar pressure patterns, measurements, and kinetics; and contextual attributes such as demographic, lifestyle, location, product preference, and style preference data.

The foot shape module 302 may be used to acquire and analyze data related to a user's foot shape. The foot shape module 302 may include one or more algorithms, such as a machine learning algorithm, that may analyze various forms of data, including but not limited to image data, to determine one or more foot shape attributes associated with a user. Foot shape attributes may include 3D models, 3D characteristics and dimensions, 2D characteristics and dimensions, and other measurements of a foot, limb, or portions thereof that may be useful to determining a user's footwear needs. In some embodiments, the raw data acquired by foot shape module 302 and the foot shape attributes associated with a user (i.e., as determined by foot shape module 302) may be stored in a personal fit database 334. For example, foot shape attributes and raw data associated with a user may be associated with a user profile in personal fit database 334. The foot shape module 302 may operate in accordance with the method described with reference to FIG. 5. It is also contemplated that, one or more pre-determined foot shape measurements and/or foot shape attributes may be received by foot shape module 302. For example, one or more foot shape measurements or foot shape attributes may have been pre-determined (e.g., in medical evaluation or other evaluation method) and stored in one or more databases that may be communicable with foot shape module 302. In this manner, the pre-determined foot shape measurements or attributes may be received without the evaluation of video or image data by the foot shape module 302.

As shown in FIG. 3, the foot shape module 302 may include one or more model reconstruction algorithms 311. The model reconstruction algorithms 311 may construct models of a user's foot, limb, or portions thereof, for example, based on image data of a user's foot, limb, or portions thereof. In some approaches, the model reconstruction algorithm 311 may be a convolutional neural network (CNN) model trained for two-dimensional (2D) to three-dimensional (3D) model reconstruction. The foot shape module may also include a reference foot model database 309. The reference foot model database 309 may include sets of 3D foot models and associated photos. In some approaches, data in the reference foot model database 309 may be used to train the model reconstruction algorithm 311.

The foot shape module 302 may also include one or more foot shape classification algorithms 310. The foot shape classification algorithms 310 may be used to determine one or more foot shape attributes associated with a user, for example, based on image data of a user's foot, limb, or portions thereof. The foot shape classification algorithms 310 may be machine learning models trained to identify one or more foot shape attributes based on imaged data and/or 3D models of a user's foot, limb, or portions thereof. The foot shape module may further include a reference foot shape category database 312 that includes foot shape attributes based on populates with shared parameters (i.e., share 3D model, image data parameters).

In operation, the foot shape module 302 may analyze foot shape data to determine at least one foot shape attribute associated with a user. Using images of a user's foot, limb, or portions thereof, the foot shape module 302 may create high quality and accurate 3D models of the user's foot. Such 3D models may help to accurately capture the complex nature of the foot's shape. The foot shape module 302 may utilize machine learning, including but not limited to neural networks, and spatial computing. It is contemplated that one or more of machine learning algorithms, such as model reconstruction algorithm 311, or spatial computing may be applied to images to assess foot dimensions and morphology.

The pathology module 304 may be used to acquire and analyze data related to foot pathologies associated with a user. The pathology module 304 may include one or more algorithms, such as a machine learning algorithm, that may analyze various forms of data, including but not limited to image data, to determine one or more foot pathology attributes associated with a user. Foot pathology attributes that may be determined using the foot pathology module may include observable foot pathologies and/or unobservable foot pathologies. In some embodiments, the raw data acquired by pathology module 304 and/or pathology attributes associated with a user (i.e., as determined by foot shape module 304) may be stored in the personal fit database 334. For example, pathology attributes and/or raw data associated with a user may be stored in a user profile in personal fit database 334.

As shown in FIG. 3, the pathology module 304 may include one or more observable pathology classification algorithms 313. The observable pathology classification algorithms 313 may include one or more machine learning models trained to identify observable foot pathologies based on image recognition. The pathology module 304 may further include one or more reference observable pathology databases 315. The reference observable pathology databases 315 may include reference image data of populations with observable foot pathologies. In some approaches, data in the reference observable pathology databases 315 may be used to train the observable pathology classification algorithms 313.

The pathology module 304 may also include one or more unobservable pathology classification algorithms 314. The unobservable pathology classification algorithms may include one or more machine learning models trained to identify unobservable foot pathologies based on image analysis. The pathology module 304 may further include one or more reference unobservable pathology databases 316. The reference unobservable pathology databases 316 may include reference image data of populations with observable foot pathologies. In some approaches, data in the reference observable pathology databases 316 may be used to train the observable pathology classification algorithms 314.

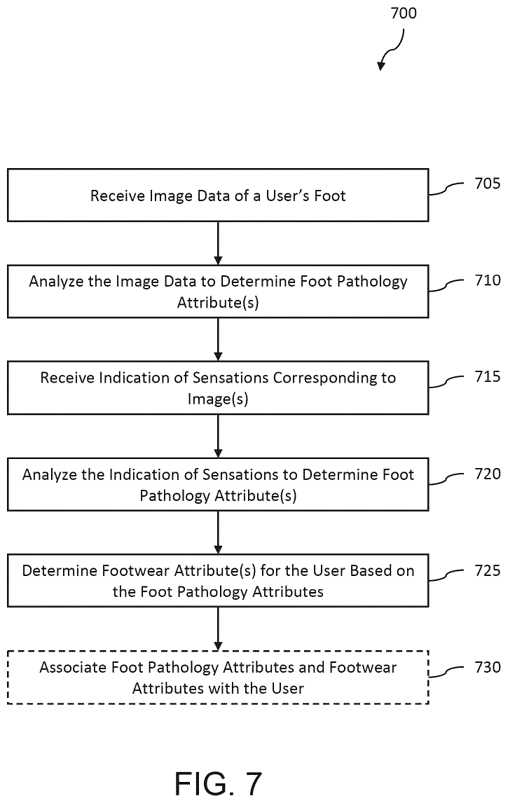

In operation, the pathology module 304 may receive data related to pathologies associated with a user. In some approaches, the pathology module 304 may acquire or otherwise receive data using an electronic user device. In one embodiment, the pathology module 304 may acquire and analyze the image data in accordance with one or more steps of the methods described with reference to FIGS. 6 and 7. In some approaches, the pathology module may identify pathology attributes that may influence a user's footwear needs. It is also contemplated that one or more pre-determined foot pathology attributes or foot pathology information may be received by pathology module 304. For example, one or more foot pathology attributes may have been pre-determined (e.g., in medical evaluation or other pathology evaluation method) and stored in one or more databases that may be communicable with pathology module 304. In this manner, the pre-determined foot pathology attributes or information may be received without the evaluation of video or image data by the pathology module 304.

The pathology module 304 may identify both observable and unobservable foot pathologies that may influence a user's footwear needs. In some approaches, the pathology module 304 may be configured to acquire or otherwise receive foot photos, sensation maps, and/or questionnaires. In one example, to acquire image data of a user's foot, the system 300 may prompt the user to take photos or video of the user's foot.

In some embodiments, image analysis may be applied by one or more processors associated with the foot pathology module 304. For example, image analysis may be applied to sensation maps to glean data on pains and sensitivities that correspond to specific foot conditions and pathologies, which ultimately influence personal footwear requirements. In an exemplary embodiment, the pathology module 304 may also analyze responses to surveys or questionnaires to glean data on pre-existing medical and/or foot conditions (e.g., diabetes, heel spur, shin splints, or other food condition.), previous injuries (e.g., stress fractures, ankle sprains, or other types of foot and leg injuries), and medical procedures (e.g., metatarsal foot surgery, bunionectomy, or other types of foot and leg medical/surgical procedures).

The gait and biomechanics module 306 may be used to acquire and analyze data related to a user's gait and biomechanics. The gait and biomechanics module 306 may include one or more algorithms, such as machine learning algorithms, that may analyze various forms of data, including but not limited to image data or video data, to determine one or more gait and biomechanics attributes associated with a user. Gait attributes may include, for example, gait patterns, characteristics, measurements, and kinematics. Biomechanics attributes may include, for example, foot plantar pressure patterns, measurements, and kinetics. In some embodiments, the raw data or information acquired by gait and biomechanics module 306 and/or the gait attributes associated with a user (i.e., as determined by gait and biomechanics module 306) may be stored in a personal fit database 334. For example, gait attributes and raw data associated with a user may be associated with a user profile in personal fit database 334.

As shown in FIG. 3, the gait and biomechanics module 306 may include one or more gait classification algorithms 318 and may include one or more biomechanics algorithms 319. The gait classification algorithms 318 may determine gait attributes associated with a user based on, for example, image data and/or video data. In some approaches, the gait classification algorithm 318 may include a machine learning model trained for tracking a user's gait based on collection of gait video analyses and associated gait attributes. The gait module 306 may further include one or more reference gait databases 320. In some approaches, the reference gait databases 320 may include gait attributes based on populations with shared parameters. The biomechanics classification algorithms 319 may determine biomechanics attributes associated with a user based on, for example, pressure sensor data. In some approaches, the biomechanics classification algorithm 319 may include a machine learning model trained for tracking a user's gait based on collection of pressure map analyses and associated biomechanics attributes. The gait and biomechanics module 306 may further include one or more reference biomechanics databases 321. In some approaches, the reference biomechanics databases 321 may include biomechanics attributes based on populations with shared parameters.

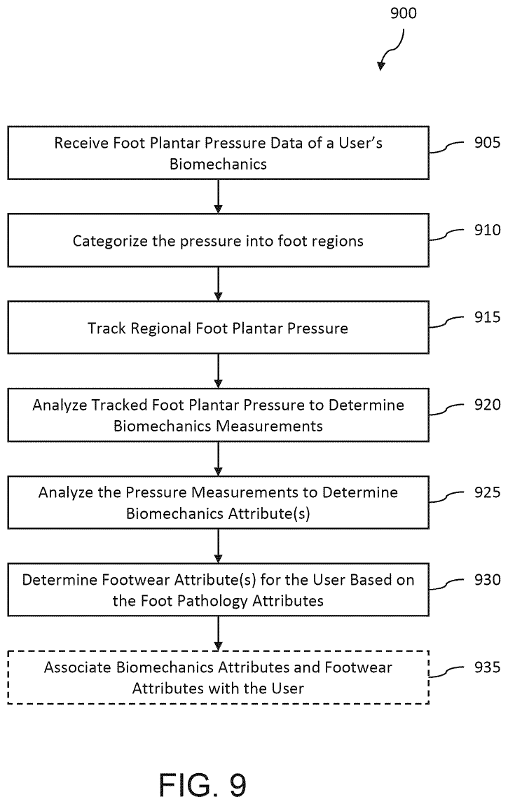

In operation, the gait and biomechanics module 306 may receive data related to the user's gait, such as image data and/or video data and user's biomechanics, such as foot plantar pressure. In one example, the gait and biomechanics module 306 may acquire and analyze the image data and/or video data to determine one or more gait attributes associated with the user in accordance with one or more steps of the method described with reference to FIG. 8. In some approaches, the gait and biomechanics module may identify gait attributes, such as gait patterns or characteristics, that may influence a user's footwear needs. In another example, the gait and biomechanics module 306 may acquire and analyze the foot plantar pressure data to determine one or more biomechanics attributes associated with the user in accordance with one or more steps of the method described with reference to FIG. 9. It is also contemplated that one or more pre-determined gait and biomechanics attributes may be received by gait and biomechanics module 306. For example, one or more gait and biomechanics measurements or gait and biomechanics attributes may have been pre-determined (e.g., in medical evaluation or other gait evaluation or biomechanical evaluation method(s)) and stored in one or more databases that may be communicable with gait and biomechanics module 306. In this manner, the pre-determined gait and biomechanics attributes may be received without the evaluation of video or image data by the gait and biomechanics module 306.

The context module 308 may be used to acquire and analyze data related to contextual attributes associated with a user. Contextual attributes associated with a user may include, for example, data or information related to demographics, lifestyle, location, product preferences, and style preferences. In some embodiments, the raw data acquired by the contextual module 308 as well as contextual attributes associated with a user (i.e., as determined by context module 302) may be stored in a personal fit database 334. For example, contextual attributes and raw data associated with a user may be associated with a user profile in personal fit database 334.

As shown in FIG. 3, the context module 308 may include one or more of a demographics database 322, a lifestyle database 324, and a location database 326. The demographics database 322 may include data on key demographic factors known to be associated with specific foot characteristics. The lifestyle database 324 may include data on lifestyle factors that may impact a user's footwear. The location database 326 may include data on location related factors that may impact a user's footwear needs.

In operation, the context module 308 may receive data from a user, for example, through surveys or questionnaires related to demographics, product preferences, lifestyle, and location (i.e., the climate or setting in which the user will wear their footwear). The context module 308 may leverage data in one or more of the databases (i.e., demographics database 322, lifestyle database 324, and location database 326) to determine one or more contextual attributes associated with a user. For example, the context module 308 may use demographics database 322 to determine a personal contextual attribute associated with the user based on demographics data. The context module 308 may use lifestyle database 324 to determine a personal contextual attribute associated with the user based on lifestyle data. The context module 308 may use location database 326 to determine a personal contextual attribute associated with the user based on location data.

FIG. 3 illustrates an exemplary data structure for foot shape module 302, pathology module 304, gait and biomechanics module 306, and context module 308. However, it is contemplated that the system 300 may utilize alternative data structures to operate the foot shape module 302, pathology module 304, gait and biomechanics module 306, and context module 308. For example, any one of these modules may be communicable with any one of the databases illustrated in the system 300. In addition, while illustrated as separate modules, it is contemplated that a single engine, processor, server, or computing device may be employed to conduct each of the collection and analysis of the foot shape, pathology, gait, biomechanics, and contextual data. Additionally, although each of the modules and databases depicted in FIG. 3 are depicted as part of single system, it should be understood that any of the modules and/or database may be housed in a separate system or on separate servers.

The system 300 may also include a personal attribute analysis module 328. The personal attribute analysis module 328 may analyze the assessment information or one or more personal attributes (i.e., foot shape attributes, pathology attributes, gait and biomechanics attributes, and/or contextual attributes) associated with a user to determine one or more footwear attributes to associate with the user. Footwear attributes may include any characteristic, feature, or product specification that may influence calculated footwear fit and/or footwear personalization. Footwear attributes may include, for example, information related to a footwear product's construction (e.g., design, seams, and dimensions), materials, materials properties (e.g., elasticity, stretchability, flexibility, breathability, waterproofing), style, appearance, assigned category, aesthetic descriptors, etc. It is also contemplated that footwear attributes may include characteristics, features, or product specifications that may influence the impact of a footwear product on skin or the interaction of multiple materials used in the footwear product.

The personal attribute analysis module 328 may include one or more classification algorithms. The classification algorithms may analyze personal attributes, or the assessment information associated with the user, to determine one or more footwear attributes to associate with a user based on the user's personal attributes or assessment information. In some approaches, the footwear classification algorithms may include one or more machine learning or CNN models to identify footwear attributes based on various inputs. Such inputs may include, but are not limited to, 3D models, foot shape attributes, pathology attributes, gait and biomechanics attributes, and/or contextual attributes. Inputs may further include any form of raw assessment information input into system 300, such as image data, video data, context data, questionnaire responses, etc.

The system 300 may also include one or more prioritized footwear classification modules 330, which may include one or more classification algorithms. The prioritized footwear classification module 330 may include one or more machine learning models trained to identify footwear attributes associated with a user based on a user's personal attributes (i.e., foot shape attributes, pathology attributes, gait and biomechanics attributes, and/or contextual attributes). In this manner, the prioritized footwear classification module 330 may identify footwear attributes that are best suited for a user, based on the user's personal attributes. The prioritized footwear classification module 330 may be communicable with one or more attribute relationship databases 332. The attribute relationship database 332 may include data on relationships between various footwear attributes and personal attributes (i.e., foot shape attributes, pathology attributes, gait and biomechanics attributes, contextual attributes).

Described below are various methods of operating the foot shape module, pathology module, gait and biomechanics module, and context module depicted in FIG. 3. These methods may be used, for example, to assess personal attributes associated with a user to provide personalized footwear. Personal attributes may assess one or more of the foot shape, pathologies, gait, biomechanics, and contextual information such as demographics, lifestyle, location, and/or product preferences of a user. Such personal attributes may influence footwear product design to produce a product that best suits the fit, comfort, and preferences of the user. One or more of the modules may be operated together to gain a comprehensive understanding of the personal attributes associated with a user. As discussed above, one or more modules may be utilized to provide inputs for the design for the personalized footwear for a user and no one module is required to execute the methods described herein. Further, as mentioned above, it is also contemplated that assessment information (i.e., foot shape, pathologies, gait, biomechanic information, etc.) is input into the design for the personalized footwear without further determination or analysis via any of the modules.

### Foot Shape Module

An exemplary method of acquiring and displaying foot shape data, in accordance with some embodiments, is described below. One or more electronic user devices, such as the electronic user devices described with reference to FIG. 3 may be used to carry out one or more steps of the method. In some approaches, the method may be executed by or in conjunction with the foot shape module 302 described with reference to FIG. 3. One or more steps of the method may be used to acquire and display foot pathology data.

The method includes acquiring or otherwise receiving image data associated with a user's foot, limb, or portions thereof. The image data may include, for example, photos, images, and/or video. In some approaches, the image data may include three photos of an individual's foot from specific angles. The image data may be acquired or otherwise captured via an image sensor, such as a camera, associated with an electronic user device. In one example, a user may take three photographs of each foot using a camera to capture the following three views: top, inner, and outer. Image data may include any number of views, for example anywhere between 1 and 20, 1 and 10, and 1 and 5 views, and may also include other views such as front, rear, and bottom. In some approaches, the camera may provide augmented reality (AR) guides to ensure the proper positioning of the camera. It is also contemplated that the method is not limited to image data but may include acquiring other forms of data representative or indicative of a user's foot, limb, or portions thereof. It is also contemplated that image data may be acquired, for example, from one or more databases.

In some approaches, the image data may capture the user's foot in relation to another object, for example, a piece of standard letter paper. In one embodiment, a user will place his/her foot on a piece of standard letter paper to obtain one or more views.

The method may also optionally include instructing a user regarding the image data acquisition. In some approaches, instructions may be transmitted to user via a user interface associated with an electronic user device, for example, before or while a user is capturing image data. The prompt may instruct the user to take a specified number of photos, to take photos from a particular view or angle, to place a particular foot, limb, or portion thereof within the view of the image sensor, to adjust lighting, where to place a foot, limb, or portion thereof relative to another object, or to adjust the position of the electronic user device being used to acquire image data.

After acquiring image data, the method may optionally include, displaying the image of the user's foot, limb, or portion thereof to a user via the user interface. In some approaches, the image may be a 3D model of the user's foot generated, at least in part, based on the received image data.

In one embodiment, the user interface may also display one or more personalized recommendations regarding one or more footwear product specifications based, at least in part, on the foot shape data. Recommendations may suggest one or more footwear products, sizes, styles, models, materials, material properties (e.g., breathability, viscoelasticity, stretchability, tear strength, flexibility, water resistance, abrasion resistance, etc.), weight, tongue feature(s), lining feature(s), insole feature(s), toe box feature(s), outsole feature(s), upper feature(s), sole feature(s), heel height, degrees of foot support, or other footwear attributes for the user.

FIG. 5 illustrates an exemplary method 500 of analyzing image data of a user's foot, limb, or portion thereof to determine foot shape information or one or more foot shape attributes associated with the user. In some embodiments, one or more steps of method 500 may be executed by or in conjunction with the foot shape module 302 described with reference to FIG. 3.

The method 500 includes receiving 505 image data of a user's foot, limb, or portion thereof. In some embodiments, the image data may include the image data acquired using the method described with reference to FIG. 4. For example, the image data may include image data of a user's foot, limb, or portion thereof from the following three views: top, inner, and outer. Image data may include any number of views, for example anywhere between 1 and 20, 1 and 10, and 1 and 5 views, and may also include other views such as front, rear, and bottom. The method then generates 510 a 3D model of the user's foot, limb or portion thereof based on the image data. In some approaches, at least one model reconstruction algorithm may be applied to the image data to generate the 3D model. In some approaches, the model construction algorithm may also extract at least one dimension of a user's foot, limb, or portions thereof from the image data and/or the 3D models. In some embodiments, the model reconstruction algorithm may be used to extract at least one foot dimension associated with the user's foot, limb, or portion thereof from the image data. In some approaches, the model reconstruction algorithm may be a CNN model trained 2D image to 3D model reconstruction. For example, the CNN model may be trained using reference sets of 3D models and associated photos from the three sets of views. In addition, the method 500 may further include determining at least one foot dimension in two dimensions using image processing, for example, based on foot layout on a sheet of paper (e.g., as captured in image data). Such dimensions may also be used to construct a 3D model of the user's foot or a portion thereof.

The method 500 may further include analyzing 520 the 3D models of the user's foot, limb, or portions thereof to determine at least one foot shape attribute associated with the user. In some embodiments, foot shape attributes may be determined by applying one or more foot shape classification algorithms to the 3D model and/or the image data. In some approaches, the foot shape classification algorithm may be a machine learning algorithm trained to identify one or more foot shape attributes based on the 3D model and/or the image data. In other embodiments, the foot shape attributes may be determined using spatial computing to assess at least one foot dimension or morphology associated with the user's foot, limb, or portion thereof using the image data.

Foot shape attributes may include any foot shape characteristics that may impact footwear. Foot shape attributes may include foot dimensions such as arch height, foot width, global foot width, midfoot width, foot length, ball width, inter-toe dimensions, ball circumference, ball angle, heel circumference, lateral metatarsal length, medial metatarsal length, arch length, instep height, and instep distance. Foot shape attributes may also include shape-related characteristics or morphologies such as hallux bone orientation, toe orientation, or foot type. By some approaches, at the foot shape attributes may include about 10, 8, 6, 4, or 2 dimensions that capture at least 75%, at least 85%, at least 90%, and in some instances about 92.6% of shape variation of the user's foot. In one example, foot shape attributes include 6 dimensions including arch height, combined ball width and inter-toe distance, global foot width, foot type, and midfoot width. In some embodiments, foot shape attributes may be determined by applying one or more foot shape classification algorithms to the 3D model and/or the image data. In some approaches, the foot shape classification algorithm may be a machine learning algorithm trained to identify one or more foot shape attributes based on the 3D model and/or the image data. In other embodiments, the foot shape attributes may be determined using spatial computing to assess at least one foot dimension or morphology associated with the user's foot, limb, or portion thereof using the image data to.

It is also contemplated that one or more foot shape measurements or foot shape attributes may be received from a database. For example, one or more foot shape measurements or foot shape attributes may have been pre-determined (e.g., in medical evaluation or other foot shape evaluation method). In this manner, the pre-determined foot shape measurements/or foot shape attributes may be received without the evaluation of video or image data. That is, steps 505-520 are not required to determine one or more foot shape measurements or foot shape attributes associated with a user.

The method 500 then includes determining 525 a footwear attribute to associate with the user based on at least one foot shape attribute or the foot shape information. Footwear attributes associated with the user may include attributes of a footwear product best suited for the user based on the user's foot shape attribute. In one example, if a foot shape attribute of the user is a high arch, the footwear attribute associated with the user may be high arch support. In another example, when a foot shape attribute associated with the user is a stacked toe or other digital deformity, the footwear attribute associated with the user may be materials with high elasticity. In some embodiments, classification algorithms may be used to identify at least one footwear attribute that is best suited the user based on the user's foot shape attributes. In some approaches, the classification algorithm may include a machine learning model trained to identify footwear attributes to associate with a user based on the user's foot shape attributes.

In some embodiments, the method 500 may include determining a footwear attribute to associate with the user using established relationships between various footwear attributes and various foot shape attributes. In one exemplary embodiment, relationships between footwear attributes and foot shape attributes may be quantified by giving a score, for example a score of 0-100, to a footwear attribute based on the foot shape attribute. A high score may indicate a strong preference for a specific parameter while a lower score indicates unsuitability for a specific parameter. The relationships between footwear attributes and foot shape attributes may be stored, for example, in a database.

The method 500 may optionally include associating 530 the foot shape attributes and/or footwear attributes with the user. In some approaches, the foot shape attributes and/or footwear may be associated with the user in a database, such as the exemplary personal fit database depicted in FIG. 3. For example, the database may include a user profile storing one or more foot shape attributes associated with the user. In some examples, the user profile may also store one or more footwear attributes associated with the user.

By acquiring and analyzing foot shape data in the above-described manner, the precise 3D characteristics of a user's foot, limb, or portions thereof may be captured and utilized to provide personalized footwear that is tailored to the user's foot shape.

### Pathology Module

FIG. 6 illustrates an exemplary method 600 of acquiring and displaying foot pathology data, in accordance with some embodiments. One or more electronic user devices, such as the electronic user devices described with reference to FIG. 3 may be used to carry out one or more steps of method 600. In some approaches, method 600 may be executed in by or in conjunction with the pathology module 304 described with reference to FIG. 3. One or more steps of method 600 may be used to acquire and display foot pathology data.

The method 600 includes acquiring 605 or otherwise receiving image data associated with a user's foot, limb, or portions thereof. The image data may include, for example, photos, images, or video. The image data may be acquired or otherwise captured via an image sensor, such as a camera, associated with an electronic user device. In some approaches, the image data may include three photos of an individual's foot from specific angles. For example, a user may take three photographs of each foot using a camera capture the following three views: top, inner, and outer. Image data may include any number of views, for example anywhere between 1 and 20, 1 and 10, and 1 and 5 views, and may also include other views such as front, rear, and bottom. Image data may be acquired using a camera having feature that provides AR guides for the proper camera positioning. It is also contemplated that method 600 is not limited to image data but may include other forms of data representative or indicative of a user's foot, limb, or portions thereof. It is also contemplated that image data may be acquired, for example, from one or more databases.

The method 600 may also optionally include providing instructions 610 to a user regarding, for example, how to acquire image data. Instructions may be provided, for example, by sending a prompt to a user via a user interface associated with the electronic user device, for example, before or while a user is capturing image data. Instructions may instruct the user to take a specified number of photos, to take photos from a particular view or angle, to place a particular foot, limb, or portion thereof within the view of the image sensor, to adjust lighting, or to adjust the position of the electronic user device being used to acquire image data.

After acquiring image data, the method 600 may also include displaying 615 the image of the user's foot, limb, or portion thereof to a user via the user interface. In some approaches, the image may be a 3D model of the user's foot generated, at least in part, based on the received image data. The method 600 also includes receiving 620 an indication of one or more sensations associated with an image of the user's foot, limb, or portion thereof. In some embodiments, a user may map sensations onto one or more images by indicating areas experiencing one or more sensations. Sensation mapping may be performed using a user interface of an electronic user device. In one approach, a user may indicate sensations on one or more images by drawing on images of the user's foot, for example, using a stylus or finger on a touch screen of the electronic user device. In some embodiments, a user may select various colors or markings to indicate various sensations associated with a foot, limb, or portion thereof that is captured in the image. Sensations may include, for example, numbness, burning, aching, pins and needles, itching, soreness, throbbing, etc. It is also contemplated that a user may map or otherwise indicate one or more injuries, medical conditions, medical procedures, etc. on the image data.

The method 600 also includes receiving 625 information regarding one or more foot pathology attributes associated with the user. In some approaches, such information may be received from a database housing one or more previously determined foot pathology attributes. In other approaches, a questionnaire may be transmitted to the user to obtain information regarding one or more foot pathology attributes. In some approaches, the questionnaire may be transmitted to an electronic user device associated with the user and displayed via the user interface of the device. The questionnaire may include one or more questions, prompts, or requests for information related to pathology attributes associated with the user. The questionnaire may pertain to any foot pathology attributes including but not limited to, pre-existing medical and foot conditions, injuries, medical procedures that impact foot function and gait, foot biomechanics, history of foot injuries, history of limb injuries, history of foot medical procedures, history of lower-limb medical procedures, history of problems or successes with other shoes, and any other foot related-medical information. In response to receiving the questionnaire, a user may input information related to the queries, prompts, or requests for information via the user interface. The method may further optionally include associating the foot pathology attributes with the user. In some approaches, the foot pathology attributes may be associated with the user in a database, such as the exemplary personal fit database depicted in FIG. 3. For example, the database may include a user profile storing one or more foot pathology attributes associated with the user.

The user interface may also display one or more personalized recommendations regarding one or more footwear product specifications based, at least in part, on the pathology data. Recommendations may suggest one more footwear products, sizes, styles, models, materials, material properties (e.g., breathability, viscoelasticity, stretchability, tear strength, flexibility, water resistance, abrasion resistance, etc.), weight, tongue feature(s), lining feature(s), insole feature(s), toe box feature(s), outsole feature(s), upper feature(s), sole feature(s), heel height, degrees of foot support, construction (i.e., stitching, mesh), or other footwear attributes for the user. The recommendations may be used to design the personalized footwear.

Ultimately, the foot pathology information and/or foot pathology attributes may be used to determine 630 the designs for the personalized modular last and any necessary personalized shoe components.

FIG. 7 illustrates an exemplary method 700 of analyzing image data to determine foot pathology attributes associated with a user. In some embodiments, one or more steps of method 700 may be executed by or in conjunction with the pathology module 304 described with reference to FIG. 3.

The method 700 includes receiving 705 image data of a user's foot, limb, or portion thereof. In some embodiments, the image data may include image data acquired using the method described with reference to FIG. 5. For example, the image data may include image data of a user's foot, limb, or portion thereof from the following three views: top, inner, and outer. Image data may include any number of views, for example anywhere between 1 and 20, 1 and 10, and 1 and 5 views, and may also include other views such as front, rear, and bottom.

The method 700 then includes analyzing 710 the image data to determine at least one foot pathology attribute, such an observable foot pathology. Such observable foot pathologies may include digital deformities (e.g., hammertoe, claw toe, mallet toe, crossover toe), hallux valgus, tailor's bunion, blisters, or other general trauma. Observable pathologies may be automatically identified, for example, based on the image data of a user's foot, limb, or portion thereof. In some embodiments, an observable pathology classification algorithm is applied to the image data to identify one or more observable foot pathologies. In some approaches, the observable pathology classification algorithm may be a machine learning model trained to identify observable foot pathologies based on image recognition. In some instances, reference images of individuals with known observable foot pathologies may be used to train the machine learning model.

The method 700 also includes receiving 715 an indication of sensations corresponding to one or more images of a user's foot. In some embodiments, such an indication may be in the form of a sensation map, for example, the sensation map described with reference to FIG. 6. The method then includes analyzing 720 the indication of sensations to determine at least one foot pathology attribute, such as an unobservable foot pathology attribute. Unobservable foot pathology attributes may include, for example, central metatarsalgia, plantar fasciitis, heel spurs, and diabetic neuropathy. It is contemplated that, by receiving an indication of sensations corresponding to areas of a user's foot, for example, by mapping sensations to image data of the user's foot, it is possible to establish correlations between sensations and unobservable pathologies that may impact footwear needs. In some embodiments, at least one unobservable foot pathology may be determined by applying an unobservable pathology classification algorithm to a sensation map or to some other indication of sensations corresponding to an image of a user's foot. In some approaches, the unobservable pathology classification algorithm may include a machine learning model trained to identify unobservable foot pathologies based on image analysis. For example, the machine learning model may be trained using reference sensation maps of feet with known unobservable foot pathologies.

It is also contemplated that one or more foot pathology attributes may be received from a database. For example, one or more foot pathology attributes may have been pre-determined (e.g., in a medical assessment or other evaluation process). In this manner, the pre-determined foot pathology data and/or foot pathology attributes may be received without the evaluation of image data and/or questionnaires. That is, steps 705-720 are not required to determine one or more foot pathology attributes associated with a user.

The method 700 then includes determining 725 a footwear attribute to associate with the user based on at least one foot pathology attribute. Footwear attributes associated with the user may include attributes of a footwear product best suited for the user based on the user's foot pathology attributes. For example, if a foot pathology attribute of the user is a diabetic neuropathy, the footwear attribute associated with the user may be a closed-toe style. In another example, if a foot pathology attribute associated with the user is a bunion (hallux valgus), tailor's bunion, or other digital deformity, the footwear attribute associated with the user may be an upper with high elasticity and/or an upper that is seam free. In another example, if a foot pathology attribute associated with the user is hyperhydrosis, the footwear attribute associated with the user may be a material with high breathability. In some embodiments, classification algorithms may be used to identify at least one footwear attribute that best suits the user based on the user's foot pathology attributes. In some approaches, the classification algorithm may include a machine learning model trained to identify footwear attributes to associate with a user based on the user's foot pathology attributes. In some embodiments, the method may determine a footwear attribute to associate with the user by using established relationships between various footwear attributes and various foot pathology attributes. In one exemplary embodiment, relationships between footwear attributes and foot pathology attributes may be quantified by giving a score, for example a score of 0-100, to a footwear attribute based on the foot pathology attribute. A high score may indicate a strong preference for a specific parameter while a lower score indicates unsuitability for a specific parameter. The relationships between footwear attributes and foot pathology attributes may be stored, for example, in a database.

The method 700 may optionally include associating 730 the foot pathology attributes and/or footwear attributes with the user. In some approaches, the foot pathology attributes and/or footwear may be associated with the user in a database, such as the exemplary personal fit database depicted in FIG. 3. For example, the database may include a user profile storing one or more foot pathology attributes associated with the user. In some examples, the user profile may also store one or more footwear attributes associated with the user.

By acquiring and analyzing pathology data in the manner described in FIGS. 6-7, observable and unobservable pathologies associated with a user's foot, limb, or portions thereof may be captured and utilized to provide personalized footwear that is tailored to the user's foot pathologies.

### Gait Module

An exemplary method of acquiring and displaying gait data, in accordance with some embodiments, is described below. One or more electronic user devices, such as the electronic user devices described with reference to FIG. 3 may be used to carry out one or more steps of the method. In some approaches, the method may be executed in by or in conjunction with the gait and biomechanics module 306 described with reference to FIG. 3. One or more steps of the method may be used to acquire and display gait and biomechanics data associated with a user.

The method includes acquiring or otherwise receiving video data associated with a user's gait. The video data may include, for example, video of the user walking. In some approaches, the video data may include two videos of the user walking, where the video is acquired from two specific views. The two specific views may be, for example, a frontal view and a side view. The video data may be acquired or otherwise captured via an image sensor, such as a camera, associated with an electronic user device. In some approaches, the video data may be markerless video data. It is also contemplated that the method is not limited to video data but may include other forms of data representative or indicative of a user's foot, limb, or portions thereof. It is also contemplated that video data may be acquired, for example, from one or more databases. Such data may be, for example, pre-recorded video or images or other pre-captured data indicative of a user's gait.

The method may also optionally include providing instructions to a user regarding, for example, how to acquire video data. Instructions may be provided, for example, by sending a prompt to a user via a user interface associated with the electronic user device, for example, before or while a user is capturing video data. Instructions may instruct the user (or an assistant to the user) to record video for a specified duration, to take video from a particular view or angle, to adjust the video background, to have the user take a specified number of steps or strides, to adjust the frame rate of the video capture, to adjust lighting, or to adjust the position of the electronic user device being used to acquire video data. After acquiring or otherwise receiving video data capturing the user's gait, the method may optionally include displaying the video of the user's gait. For example, the video may be displayed on a user interface of an electronic user device.

After acquiring video data, the method may also optionally include displaying one or more footwear products, for example, through an electronic product catalogue. The user may view the footwear products on the user interface of the electronic user device. The product catalog may include various footwear products, models and/or styles. A user may have the option to select one or more of the footwear products being viewed, for example, through a user interface of the electronic device. The user may then select a particular footwear product, model, or style.

The user interface may also display one or more personalized recommendations regarding one or more footwear product specifications, based at least in part, on the gait and biomechanics data. Recommendations may suggest one more footwear products, sizes, styles, models, materials, material properties (e.g., breathability, viscoelasticity, stretchability, tear strength, flexibility, water resistance, abrasion resistance, etc.), weight, tongue feature(s), lining feature(s), insole feature(s), toe box feature(s), outsole feature(s), upper feature(s), sole feature(s), heel height, degrees of foot support, construction (i.e., stitching, mesh), or other footwear attributes for the user. The recommendations may be used to design the personalized footwear.

FIG. 8 illustrates an exemplary method 800 of performing a gait assessment, in accordance with some embodiments. In some embodiments, one or more steps of method 800 may be executed by or in conjunction with the gait and biomechanics module 306 described with reference to FIG. 3.

The method 800 includes receiving 805 video data of a user's gait. In some embodiments, the video data may include the image data acquired using the method described above. For example, the video data may include a video of the user walking. In some approaches, the video data may include two videos of the user walking, where the video is acquired from two specific views. It is contemplated that the video data may be markerless video, in which no distinct object is placed on the region of interest (e.g., the limb or foot) for the purpose of being used for tracking. In some examples, anatomical features, personal features, skin tones, and deformities are excluded from being considered as intentional markers. The video data may then be parsed 810 into frames.

The method 800 further includes tracking 815 at least one limb of the user. For example, one or more points on a limb of the user may be tracked. Points that may be tracked may include one or more of a lateral knee condyle, a patella (i.e., knee-cap), a proximal tibia, a distal tibia, an ankle joint, an ankle joint including malleolus, a foot, a foot including navicular, and a toe. In some embodiments, tracking may be markerless, or tracked without the user of reference markers in the view of the video. In some approaches, tracking may be performed using one or more tracking-based machine learning algorithms to identify key points on a limb of the user. In some approaches, tracking involves tracking the motion of one or more points between frames of the video.

The method 800 may further include analyzing 820 tracked limbs to determine at least one gait attribute, such as a kinematic gait measurement. Kinematic gait measurements may include, for example, step time (i.e., time duration between ipsilateral and contralateral heel strike events), step length (i.e., horizontal distance traveled by heel between ipsilateral and contralateral heel strikes), gait speed (i.e., step length divided by step time), stance time (i.e., time between consecutive heel strike and toe off events), and stance swing (i.e., time between toe off and the consecutive heel strike event). Gait attributes may also include foot position, foot plantar flexion, ankle plantar flexion, joint loading, foot loading, and other lower-limb or foot kinetics. Tracked points on video frames may provide detailed information about gait. In some approaches, motion analysis may be applied to tracked points on video frames to assess gait.

The method 800 may also further include analyzing 825 kinematic gait measurements to determine at least one gait attribute. Gait attributes may include any gait categories, patterns, or characteristics which may inform a user's footwear needs. In some embodiments, kinematic gait measurements may be determined using one or more gait classification algorithms. In some approaches, the gait classification algorithms may include one or more machine learning models trained for tracking points based on one or more of gait video, gait patterns, gait measurements, and gait characteristics. In some examples, the machine learning model may be trained using reference gait data from populations with known gait parameters.

It is also contemplated that one or more gait measurements or gait attributes may be received from a database. For example, one or more gait measurements or gait attributes may have been pre-determined (e.g., in a gait lab or through another gait evaluation method). In this manner, the pre-determined gait attributes and/or gait attributes may be received without the evaluation of video or image data. That is, steps 805-825 are not required to determine one or more gait measurements or gait attributes associated with a user.

The method 800 may also include determining 830 a footwear attribute to associate with the user based on at least one gait attribute. Footwear attributes associated with the user may include attributes of a footwear product best suited for the user based on the user's gait attributes. For example, if a gait attribute of the user is under pronation, the footwear attribute associated with the user may be high arch support. In some examples, gait attributes such as heel strike, forefoot strike, or midfoot strike may implicate cushioning and/or sole thickness (i.e., footwear attributes). Accordingly, in some examples, for gait attributes like heel strike, forefoot strike, or midfoot strike, footwear attributes related to cushioning and/or sole strike may be associated with a user. In some embodiments, classification algorithms may be used to identify at least one footwear attribute that best suits the user based on the user's gait attributes. In some approaches, the classification algorithm may include a machine learning model trained to identify footwear attributes to associate with a user based on the user's gait attributes.

In some embodiments, the method 800 may determine a footwear attribute to associate with the user using established relationships between various footwear attributes and various gait attributes. In one exemplary embodiment, relationships between footwear attributes and gait attributes may be quantified by giving a score, for example a score of 0-100, to a footwear attribute based on the gait attribute. A high score may indicate a strong preference for a specific parameter while a lower score indicates unsuitability for a specific parameter. The relationships between footwear attributes and gait attributes may be stored, for example, in a database.

The method 800 may optionally include associating 835 the gait attributes and/or footwear attributes with the user. In some approaches, the gait attributes and/or footwear may be associated with the user in a database, such as the exemplary personal fit database depicted in FIG. 3. For example, the database may include a user profile storing one or more gait attributes associated with the user. In some examples, the user profile may also store one or more footwear attributes associated with the user.

By acquiring and analyzing gait data in the above-described manner, gait attributes associated with a user's foot, limb, or portions thereof may be captured and utilized to provide personalized footwear that is tailored to the user's gait.

FIG. 9 illustrates an exemplary method 900 of performing a biomechanics assessment, in accordance with some embodiments. In some embodiments, one or more steps of the gait and biomechanics module 306 described with reference to FIG. 3 may be executed.

The method 900 includes receiving 905 foot plantar pressure data of a user. In some embodiments, the pressure data may include the pressure color map data and/or numerical pressure data acquired using the sensor. For example, the pressure data may correspond to a user standing or walking. In some approaches, the pressure data may include one or both feet of a user walking. It is contemplated that the pressure data may be in numerical, color map, and/or waveform and may be collected from one or more sensors or individual sensing units simultaneously. The pressure data may then be categorized 910 into distinct foot regions which may include forefoot, midfoot, and backfoot and may be further categorized into medial and lateral aspects.

The method 900 further includes tracking 915 at least one region of the foot plantar pressure either in real time or after recording. For example, one or more points on a sensor on one or both feet of the user may be tracked. Points that may be tracked may one or more locations in one or more of the regions 910 of the foot. In some embodiments, tracking will be assisted by the location of the sensing unit on the sensor. In some approaches, tracking may be performed using one or more tracking-based machine learning algorithms.

The method 900 may further include analyzing 920 tracked limbs to determine at least one biomechanics attribute, such as a foot plantar pressure measurement. Biomechanics attributes may also include foot position, foot plantar flexion, ankle plantar flexion, joint loading, foot loading, and other lower-limb or foot kinetics. In some approaches, motion analysis may be applied to tracked points on video frames to assess gait.

The method 900 may also further include analyzing 925 biomechanics pressure and/or other related measurements to determine at least one biomechanics attribute. Biomechanics attributes may include any biomechanics categories, patterns, or characteristics which may inform a user's footwear needs. In some embodiments, biomechanics measurements may be determined using one or more biomechanics classification algorithms. In some approaches, the biomechanics classification algorithms may include one or more machine learning models trained for tracking points based on one or more of pressure color maps, pressure values, sensor units, foot regions, and foot postures. In some examples, the machine learning model may be trained using reference biomechanics data from populations with known biomechanics parameters.

It is also contemplated that one or more biomechanics measurements or biomechanics attributes may be received from a database. For example, one or more biomechanics measurements or biomechanics attributes may have been pre-determined (e.g., in a biomechanics lab or through another biomechanics evaluation method). In this manner, the pre-determined biomechanics attributes and/or biomechanics attributes may be received without the evaluation of pressure sensor or other sensor data. That is, steps 905-925 are not required to determine one or more biomechanics measurements or biomechanics attributes associated with a user.

The method 900 may also include determining 930 a footwear attribute to associate with the user based on at least one biomechanics attribute. Footwear attributes associated with the user may include attributes of a footwear product best suited for the user based on the user's biomechanics attributes. For example, if a biomechanics attribute of the user is under pronation, the footwear attribute associated with the user may be high arch support. In some examples, biomechanics attributes such as heel strike, forefoot strike, or midfoot strike may implicate cushioning and/or sole thickness (i.e., footwear attributes). Accordingly, in some examples, for biomechanics attributes like heel strike, forefoot strike, or midfoot strike, footwear attributes related to cushioning and/or sole strike may be associated with a user. In some embodiments, classification algorithms may be used to identify at least one footwear attribute that best suits the user based on the user's biomechanics attributes. In some approaches, the classification algorithm may include a machine learning model trained to identify footwear attributes to associate with a user based on the user's biomechanics attributes.

In some embodiments, the method 900 may determine a footwear attribute to associate with the user using established relationships between various footwear attributes and various biomechanics attributes. In one exemplary embodiment, relationships between footwear attributes and biomechanics attributes may be quantified by giving a score, for example a score of 0-100, to a footwear attribute based on the biomechanics attribute. A high score may indicate a strong preference for a specific parameter while a lower score indicates unsuitability for a specific parameter. The relationships between footwear attributes and biomechanics attributes may be stored, for example, in a database.

The method 900 may optionally include associating 935 the biomechanics attributes and/or footwear attributes with the user. In some approaches, the biomechanics attributes and/or footwear may be associated with the user in a database, such as the exemplary personal fit database depicted in FIG. 3. For example, the database may include a user profile storing one or more biomechanics attributes associated with the user. In some examples, the user profile may also store one or more footwear attributes associated with the user.

### Context Module

An exemplary method of acquiring and displaying contextual data associated with a user, in accordance with some embodiments, is described below. The contextual data may include information regarding at least one personal contextual attribute associated with the user. Contextual attributes may include, for example, demographic information, product preference information, lifestyle information, location information, user footwear needs, and any other information that may impact the footwear needs of a user. One or more electronic user devices, such as the electronic user devices described with reference to FIG. 3 may be used to carry out one or more steps of the method. In some approaches, the method may be executed in by or in conjunction with the context module 308 described with reference to FIG. 3. One or more steps of the method may be used to acquire and display contextual data associated with a user.

The method includes receiving demographic data associated with a user. Demographic data may include information related to age, sex, gender, nationality, ethnicity, age, height, weight, body mass index (BMI) key variables known to be associated with specific foot characteristics (e.g., foot shape, bone structures, foot posture (including pronation/supination), and foot deformities (including digital deformities), or any other demographic factors that may impact footwear needs. In one example, a high BMI may be associated with increased ankle width, increased Achilles' tendon width, increased heel width, and a thicker forefoot along the dorsoplantar axis. In another example, age may be associated with heel width, Achilles' tendon width, toe height, and hallux orientation.

The method also includes receiving footwear preference data associated with a user. Footwear preference data may include information related to footwear style, color, brand, material, or any other factors that may impact footwear selection, design, or recommendations for a user. In one example, a user may provide information on the footwear category the user is interested in or their style preference(s). Footwear product categories may include but are not limited to: athletic shoes, casual walking shoes, boat shoes, boots/booties, clogs/mules, fashion sneakers, flats, heels, loafers, oxfords/derbys, sandals, and slippers. Style preferences may be based on footwear type and category. For style preferences, a user may be presented with a collection of images of products of that type/category that represent a broad range of style. A user may be prompted to select one or more images that match their personal style and fashion preference.

The method also includes receiving lifestyle data associated with a user. Lifestyle data may include information related to activity level, frequency of various activities, profession, social interests, hobbies, interests, sports, exercise classes, or other lifestyle factors that may impact footwear needs. In some approaches, a user may be prompted to provide information on their lifestyle, including physical activity levels or average daily step count, which may impact footwear construction, material, and durability requirements. Certain lifestyle information may be useful contextual information for assessing footwear needs and providing appropriate recommendations and fit for a user. For example, the frequency of sport activity has been associated with Achilles' tendon width and toe height. It is also contemplated that lifestyle information may be automatically received from one or more biometric sensors associated with the user. Biometric sensors may be present, for example, in a smart watch, fitness tracker, or electronic user device.

The method further includes receiving location data associated with a user. Location data may include information related to a country, city, state, county, geographic region, climate, address, zip code, or any other location-related factors that may impact a user's footwear needs. In an exemplary embodiment, the user be prompted to provide location information, such as, their residence zip code. The climate where a user resides, for example, may impact their footwear breathability and/or waterproofing requirements. It is also contemplated that location data may be automatically received from one or more sensors, such as a GPS sensor associated with a user.

It is contemplated that contextual data may be received by transmitting one or more questionnaires to a user, the questionnaires may pertain to one or more of the categories of contextual data (i.e., demographic, product preference, lifestyle, location, etc.). The questionnaire may be transmitted to an electronic user device associated with the user and displayed via the user interface of the device. The questionnaire may include one or more questions, prompts, or requests for information related to demographic information, footwear categories the user is seeking, footwear preferences, lifestyle information, and location information associated with the user. In response to receiving the questionnaire, a user may input information related to the queries, prompts, or requests for information via the user interface. In some approaches, the information may be in the form of responses or answers to the questions, prompts, or requests for information included in the questionnaire.

It is also contemplated that, one or more of demographic information, product preference information, lifestyle information, location information, or user footwear needs, or may be received from a database. For example, one or more gait measurements or gait attributes may have been pre-determined (e.g., in previous survey or personal evaluation). In this manner, the pre-determined information may be received without a questionnaire.

The method may also optionally include displaying one or more footwear products, for example, through an electronic product catalogue. The user may view the footwear products on a user interface of the electronic user device. The product catalog may include various footwear products, models and/or styles. A user may have the option to select one or more of the footwear products being viewed, for example, through a user interface of the electronic device. The user may then select a particular footwear product, model, or style.

The user interface may also display one or more personalized recommendations regarding one or more footwear product specifications, based at least in part, on the contextual data. Recommendations may suggest one more footwear products, sizes, styles, models, materials, material properties (e.g., breathability, viscoelasticity, stretchability, tear strength, flexibility, water resistance, abrasion resistance, etc.), weight, tongue feature(s), lining feature(s), insole feature(s), toe box feature(s), outsole feature(s), upper feature(s), sole feature(s), heel height, degrees of foot support, construction (i.e., stitching, mesh), construction (i.e., stitching, mesh), or other footwear attributes for the user. The recommendations may be used to design the personalized footwear.

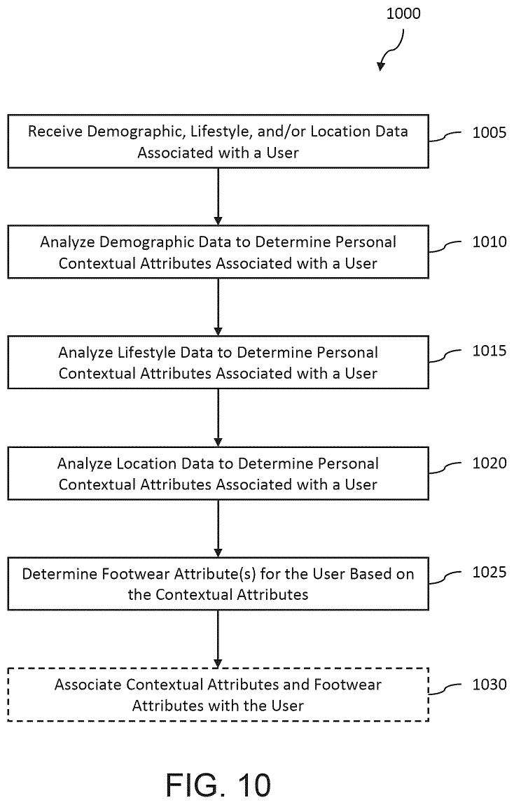

FIG. 10 is a flow diagram of a method 1000 of analyzing contextual data, in accordance with some embodiments. In some embodiments, method 1000 may be executed by or in conjunction with the context module described with reference to FIG. 3.

The method 1000 may include receiving 1005 contextual data associated with a user, such contextual data may include demographic, lifestyle, and/or location data associated with the user. In some approaches, the contextual data may be received as described above In one example, the contextual data may be received in the form of responses to a questionnaire completed by the user, in which the user is prompted to provide one or more of demographic, lifestyle, and location data.

The method 1000 may further include analyzing 1010 demographic data to determine at least one contextual attribute associated with the user. Personal contextual attributes may include any personal attributes, features, or characteristics that are related to or impacted by contextual data such as demographics, lifestyle, or location. In some approaches, the demographic data may be analyzed by a computational engine, such as the personalized footwear design and/or production engine 336 described with reference to FIG. 3. The computational engine may access a database housing reference demographic data to determine one or more contextual attributes to associate with the user based on the demographic data. The reference demographic data may include data on key contextual attributes known to be associated with specific demographic factors. Personal contextual attributes such as ankle width, Achilles' tendon width, heel width, forefoot thickness along the dorsoplantar axis, hallux orientation, and hallux varus may be associated with demographic data such as BMI results, age, or sex. Accordingly, one or more personal contextual attributes may be determined based on demographic data associated with a user, for example, by leveraging known relationships between personal contextual attributes and demographic factors. In some approaches, statistical analysis may be applied to demographic data to extract demographic-related contextual attributes associated with the user.

In one example, higher BMI may result in increased ankle width, Achilles' tendon width, heel width, and a thicker forefoot along the dorsoplantar axis. Accordingly, a user may be determined to have increased ankle width, Achilles' tendon width, heel width, or a thicker forefoot along the dorsoplantar axis when demographic data indicates the user has a high BMI. In another example, age may be related to Achilles' tendon width, heel width, toe height, and hallux orientation. Accordingly, a user may be determined to have particular Achilles' tendon width, heel width, toe height, and hallux orientation attributes when demographic data provides information on the user's age. In another example, sex may be related to ankle width, Achilles' tendon weight, and heel width. Accordingly, a user may be determined to have particular to ankle width, Achilles' tendon weight, and heel width attributes when demographic data provides information on the user's sex.

The method 1000 may also include analyzing 1015 lifestyle data to determine at least one contextual attribute associated with a user. In some approaches, the demographic data may be analyzed by a computational engine, such as the personalized footwear design and/or production engine 336 described with reference to FIG. 3. The computational engine may access a database housing reference lifestyle data to determine one or more contextual attributes to associate with the user based on the lifestyle data. The reference lifestyle data may include data on key contextual attributes known to be associated with specific lifestyle factors. Personal contextual attributes such as ankle width, Achilles' tendon width and toe width may be associated with the frequency of physical activity levels, such as the frequency of sport related activity. Accordingly, one or more personal contextual attributes may be determined based on lifestyle data associated with a user, for example, by leveraging known relationships between personal contextual attributes and lifestyle factors. In some approaches, statistical analysis may be applied to lifestyle data to extract demographic-related contextual attributes associated with the user.

The method 1000 may further include analyzing 1020 location data to determine at least one contextual attribute associated with a user. In some approaches, the location data may be analyzed by a computational engine, such as the personalized footwear design and/or production engine 336 described with reference to FIG. 3. The computational engine may access a database housing reference location data to determine one or more contextual attributes to associate with the user based on the location data. The reference location data may include data on key contextual attributes known to be associated with specific location factors. Personal contextual attributes such as preferences for footwear material, material breathability, style, and waterproofing may be associated with the climate. Accordingly, one or more personal contextual attributes may be determined based on location data associated with a user, for example, by leveraging known relationships between personal contextual attributes and location factors. In some approaches, statistical analysis may be applied to location data to extract demographic-related contextual attributes associated with the user.

It is also contemplated that one or more contextual attributes may be received from a database. For example, one or more pieces of contextual data and/or contextual attributes may have been pre-determined (e.g., in previous survey or personal evaluation). In this manner, the pre-determined information or attributes may be received. That is, steps 1005-1020 are not required to determine one or more contextual attributes associated with a user.

The method may also include determining 1025 a footwear attribute associated with the user based on at least one contextual attribute. Footwear attributes associated with the user may include attributes of a footwear product best suited for the user based on the user's contextual attributes. In some embodiments, classification algorithms may be used to identify at least one footwear attribute that best suits the user based on the user's contextual attributes. In some approaches, the classification algorithm may include a machine learning model trained to identify footwear attributes to associate with a user based on the user's contextual attributes. The method may determine a footwear attribute to associate with the user using established relationships between various footwear attributes and various contextual attributes. In one exemplary embodiment, relationships between footwear attributes and contextual attributes may be quantified by giving a score, for example a score of 0-100, to a footwear attribute based on the contextual attribute. A high score may indicate a strong preference for a specific parameter while a lower score indicates unsuitability for a specific parameter. The relationships between footwear attributes and contextual attributes may be stored, for example, in a database that may be accessed by the context module.

The method may optionally include associating 1030 the contextual attributes and/or footwear attributes with the user. In some approaches, the contextual attributes and/or footwear attributes may be associated with the user in a database, such as the exemplary personal fit database depicted in FIG. 3. For example, the database may include a user profile storing one or more contextual attributes associated with the user. In some examples, the user profile may also store one or more footwear attributes associated with the user.

### Designing Personalized Footwear

It is further contemplated that personalized footwear is designed based on the assessment information associated with the user and then manufactured. This may be initiated via the personalized footwear design and/or production engine 336. It will be appreciated that in some embodiments, the engine is a personalized footwear design engine; that is, only used in the design of the product. In other embodiments, the engine is a personalized footwear production engine; that, is only used in the production or manufacturing of the product. In yet other embodiments, the engine is a personalized footwear design and production engine; that is, used in both the design of the product and the production/manufacturing of the product. In another approach, there may be multiple engines, for example one being a personalized footwear design engine and another being a personalized footwear production engine.

As illustrated in FIG. 3, the system 300 may include a modular last design program 370 that accesses a digital library 372 of modular core last model components and a personalized shoe component design program 390 that accesses digital pattern libraries 392 to design the personalized footwear. It is further contemplated that these programs may be combined into a single program or are otherwise communicable relative to one another.

Though other ways of initiating the design are contemplated, the personalized footwear design and/or production engine 336 may initiate the modular last design program and/or the personalized shoe component design program to design a personalized modular last and/or personalized shoe components for a user based on assessment information 357 associated with the user which, as described above, may include foot shape information, pathology information, gait information, biomechanics information, and/or contextual information. In some embodiments, the assessment information is associated with a user in a user profile. As mentioned above, the assessment information associated with a user received by the system and, in some embodiments, stored in a user profile, may be directly input into the design programs in order to provide personalized designs based at least in part on the assessment information. Alternatively, at least part of the assessment information may be further analyzed to determine personal attributes and/or footwear attributes associated with the user, as described in the foregoing, and the personalized designs may be based at least in part on the personalize attributes and/or footwear attributes associated with the user. The determination of personal attributes may be performed via one or more of the above-described foot shape module, foot pathology module, gait and biomechanics module, and context module, though determination by other means is also contemplated.

Regardless of whether the design programs receive input of the user's assessment information directly or receive the user's personal attributes and/or footwear attributes derived from the user's assessment information, the input information includes at least one foot measurement (or, alternatively, a foot measurement via a shoe size, such as a standard shoe size) associated with the user so that the sizing of the personalized modular last can be determined. For example, at minimum, a foot length may be received, or both a foot length and a foot width, to determine the sizing of the personalized modular last, and such measurements may be received for both a right foot and a left foot. Foot dimensions may specifically include, for example, one or more of arch height, foot width, global foot width, midfoot width, foot length, ball width, inter-toe dimensions, ball circumference, ball angle, heel circumference, lateral metatarsal length, medial metatarsal length, arch length, instep height, and instep distance. For close alignment of the design with a user's feet, it is generally helpful to receive, for each foot, a foot length, foot width, ball width, arch height, big toe height, instep height, arch length, heel width, arch height index, heel diagonal, ball girth, arch girth, instep girth, and foot waist girth. In addition, at minimum, a shoe size might be received, which may be associated with certain measurements, and the personalized shoe last may be designed based on the shoe size or the measurements associated with the shoe size. It is further contemplated that the received measurements associated with a user may be a size or measurements associated with a size from a specific shoe from a particular footwear brand (or multiple shoes from different brands). For example, a personalized modular last may be designed based on the dimensions of a size and shoe from a brand that fits a user well.

In addition to foot measurements, the received assessment information may include any type of information or attributes that may bear on any aspect of the design for the personalized footwear (including the personalized modular last and/or any personalized shoe components), including but not limited to the above-described foot shape information or attributes, the above-described pathology information or attributes, the above-described gait information or attributes, the above-described biomechanics information or attributes, and the above-described contextual information or attributes. The information may be received, and optionally may be analyzed, in accordance with any of the above-described methods, such as those illustrated in FIGS. 5-10, or may be collected and/or received by other means.

For example, any of the above-mentioned foot measurements, or other aspects of foot shape or geometry (as described above) may be collected and/or assessed via the foot shape module described above or any of the methods and/or functions associated therewith. Foot measurements may also include measurements acquired manually, acquired through use of a Brannock Device, or may be collected or received via any other device able to provide such measurements, such as 3D scanning equipment. Measurements or other foot shape data may be received and/or input in any context, such as in the context of a medical evaluation, a shoe store evaluation, self-reporting, or any other foot shape evaluation.

Further, as an example, any pathology information or pathology attributes as described herein may collected and/or assessed via the pathology module described above or any of the methods and/or functions associated therewith. Pathology information or pathology attributes may also be acquired and/or input through different means of assessment, such as in the context of a medical evaluation, a shoe store evaluation, self-reporting, or any other pathology evaluation.

As another example, any gait and biomechanics information or attributes as described herein may be collected and/or assessed via the gait and biomechanics module described above or any of the methods and/or functions associated therewith. Gait and biomechanics information or attributes may also be acquired and/or input through different means of assessment, such as in the context of a medical evaluation (e.g., a gait laboratory), a shoe store evaluation (an observational assessment), self-reporting, or any other gait and biomechanics evaluation.

Contextual information as described herein may be collected and/or assessed via the context module as described above or any of the methods and/or functions associated therewith. Contextual information or attributes may also be acquired and/or input through different means of assessment, such as in the context of a medical evaluation, a shoe store evaluation, self-reporting, or any other contextual evaluation.

FIGS. 1, 2, 4, and 12 illustrate exemplary methods for designing a personalized footwear product, designing a personalized modular last, and/or providing a personalized footwear product. While the below description will focus largely on method 100 of FIG. 1, it will be appreciated that the description equally applies to methods 200, 400, and 1200 where the methods share the same steps.

### Designing a Personalized Modular Last

In method 100, after an initial step 105 in which assessment information associated with a user is received, including at least one foot measurement, the assessment information is used to design a personalized modular last for the user.

A personalized modular last can be an essential key to providing personalized footwear. As mentioned, a shoe last is a mechanical form, often made of wood, metal, or plastic, that is shaped like a human foot and is used to set the size, silhouette, and shape of the shoe during construction prior to being removed at the end of the shoe-making process. If a footwear product is to be personalized, focusing on personalizing the last ensures that the very foundation of the footwear product closely matches the user. Further, the last being a modular last, that is, including or being able to comprise multiple discrete components that may be combined or interchanged to create different last shapes or designs, permits more precise personalization to align with a user's footwear needs. The personalized modular last disclosed herein, and particularly the modular core last components described below, may be made of any suitable material for lasts known in the art, including plastics (such as high-density plastics), wood, or metals, for example, or combinations of such materials. It is also contemplated that the material for the modular core last components may vary depending on the type of shoe last. For example, a standard athletic shoe, with a floating lace-up tongue and a low vamp point, may use a solid last. On removing the last, the flexible shoe, typically with a bendable EVA and rubber sole, can be unlaced and easily pulled off the last without damaging the shoe. In contrast, shoes with higher vamps such as oxfords or boots may require a last material that has an ability to articulate or flex so that the uppers and sole are not damaged when the last is removed. In some embodiments, the modular lasts disclosed herein may comprise modular components that may be easy to take apart and removed from the shoe after construction, and/or may employ mechanisms for removability known in the art (e.g., a v-hinged last, a sliding hinged last, a telescoping last, etc.).

Designing the personalized modular last may include creating a digital design for a personalized modular last. A design program, such as the modular last design program 370 of system 300, may be used to perform this function. In some approaches, the digital design includes a 3D model of a personalized modular last, though 2D digital drawings are also contemplated. The digital model or drawing may be created by a computer aided design (CAD) program, however other known forms of digital drawings and design programs known in the art are also contemplated herein.

In a first step 110 of designing a personalized modular last, one or more modular core last model components are selected from a digital library 372 of modular core last model components, based at least in part on the assessment information associated with the user, which includes at least one or more foot measurements. The digital library 372 may comprise or be linked to one or more databases. In some exemplary embodiments, the modular core last model components that are available for selection have physical pre-manufactured modular core last component counterparts 369 that can be readily provided and/or assembled based on the design for the personalized modular last in case the personalized footwear product is manufactured. However, in some embodiments, one or more modular core last model components that are available to be selected may not have pre-manufactured physical modular core last component counterparts. In such a case, the physical modular core last components counterparts would need to be manufactured if a personalized footwear product is to be manufactured based on a design for a personalized modular last with one or more of such modular core last model components.

Generally speaking, the library 372 accesses a spectrum of modular core last model components which can be selected to compose a core of a personalized modular last design. The modular core last model components included in the library may be configured to cover a wide range of different foot sizes and shapes for a wide range of different shoe types or silhouettes. For example, for a certain style of running sneaker, there may be dozens or more modular core last model components that cover a vast ground of different lengths, widths, or length/width combinations. The number of modular core last model components in the library 372 is not particularly limited, though it is contemplated that in some embodiments the number advantageously strikes a balance between precise fit and scalability. That is, it may include a range of modular core lasts that is wide enough to permit a close match for many potential users, but not too wide that it would be too costly or otherwise impracticable for the range of physical core lasts to be pre-manufactured, distributed, and stored, for example, at one or more manufacturing facilities.

For example, a core last library may be based on standard shoe sizing, and may, for instance, include a certain number of different modular core lasts or last components for each half size. For example, there may be at least two, at least three, at least four, or at least 5 modular core lasts. For example, for a size 8, which corresponds to a specific length, there may be five core lasts corresponding to 5 different widths. In some embodiments, each half size may include even more core lasts or core last components. For example, in one embodiment the library can include at least ten core lasts each half size. Further, a core last library does not have to be based on and/or classified in terms of standard shoe sizing, but may use other sizing systems.

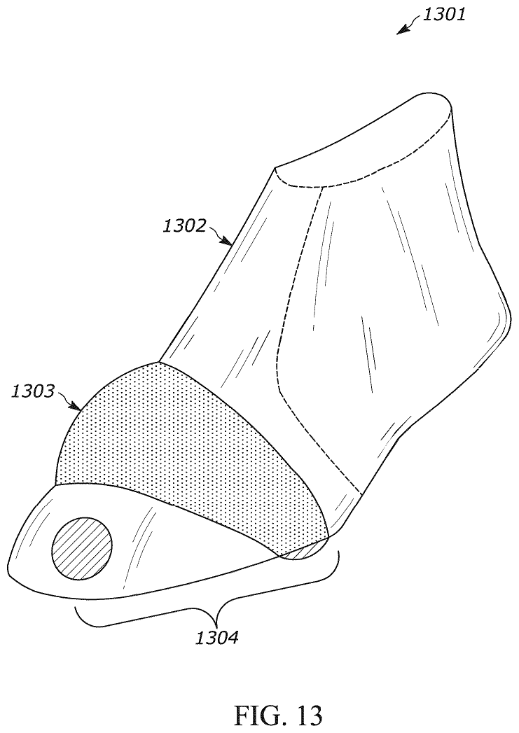

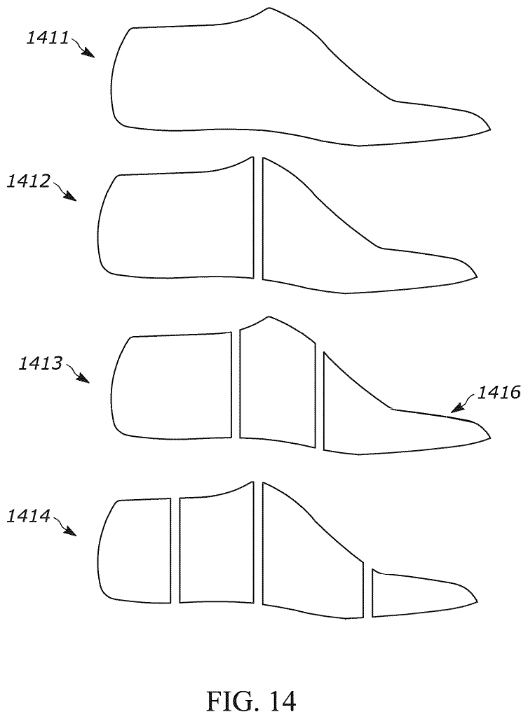

Further, as mentioned above, one or more modular core last model components may be selected to compose the design for the modular core last. That is, it is contemplated that the personalized modular last disclosed herein may have a core that comprises only one component, or may have a core that comprises multiple components. FIG. 13 illustrates an example of a personalized modular last 1301 with a modular core 1302 that is unary, comprising only a single component. Further, as illustrated in FIG. 14, a modular core may be a unary core 1411, but in alternative embodiments may be a binary core 1412 having two components 1416, a ternary core 1413 having three components 1416, or a quaternary core 1414 having four components 1416. A core comprising more than four components 1416 is also contemplated.

A core comprising multiple components may have components in different configurations to permit shape customization at different areas of the last. As illustrated in FIG. 14, an exemplary binary core last may have a core forefoot component and a core heel component. An exemplary ternary core last may have a core forefoot component, a core midfoot component, and a core heel component. An exemplary quaternary core last may have a core toe tip component, a core ball component, a core midfoot component, and a core heel component. However, other configurations are possible. For instance, FIG. 14 illustrates components having different length sections of the core last, but some embodiments may include components having different height sections or width sections of the core last.

For modular core lasts with two or more core components, the core components are configured to be removably coupled together. In one embodiment, the core components may be interlocking pieces which mate together. Other mechanisms for coupling may include mechanical fasteners, hook and loop fasteners, apertures for pins, screws, and snap-on extensions, Velcro attachments, magnets, or removable adhesive. In some exemplary embodiments, the core components are easily uncoupled so that the core components can be re-used in different configurations for different personalized lasts.

As mentioned above, designing a personalized modular last for a user includes selecting one or more modular core last model components from a digital library 372 of modular core last model components. The selection is based at least in part on the foot measurements of the user, and may include other foot shape, pathology, gait, biometrics, or contextual information associated with the user. In some exemplary embodiments, the selection process identifies a core last model in the library 372 that is a closest match to the user's foot measurements, or is a closest match in view of a combination of the user's foot measurements and other assessment information. For example, a core last model may be selected that includes a length and a width that closely approximate a length and a width of a user's foot. In another example, a core last model may be selected that includes a close match to a length of a user's foot and an arch height.

A core last comprising more than one component advantageously allows for the last to be more precisely personalized to a user's measurements. For example, if two components are selected to design a binary modular core last, the two components may be separately selected to match the unique contour of an individual's foot in a way that may not be possible with a unary core last. For example, if the user has a combination of a wider forefront and a narrower heel, one size core forefront component can be selected along with a different size core heel component. By “mix-and-matching” different core last components in this manner, the design for the core last is optimized to closely approximate the user's foot shape.

The design program may leverage algorithms to select or determine the one or more modular core last model components that is a best or closest match to the foot measurements of a user, and possibly other assessment information associated with the user. One or more machine learning algorithms may be used which may have been trained to use a wide variety of data from a user, including any assessment information, personal attributes, or footwear attributes associated with a user, to, for example, classify different core last model components and predict best matches for the user among the available core last model components. Machine learning algorithms that may be used include but are not limited to convolution neural networks (CNN, R-CNN, YOLO), Statistical Shape Analysis (SSA), Support Vector Machine (SVM), Principal Component Analysis (PCA), Bayesian networks, decision trees, nearest neighbor searching, and more.

After selecting a modular core last model, the system is configured to determine whether to include one or more last extensions in the design based on the foot measurements of the user and/or other assessment information associated with the user. It is contemplated that some users will have an excellent match to a determined core last model without any additional last extensions, so in such a case the system will determine that no last extensions are required. However, some users may have foot measurements or other foot requirements that may benefit from extending the last in one or more regions. As such, an optional second step 115 of designing a personalized modular last includes determining one or more last extensions that may be added to the last design.

In some embodiments, the shape, dimensions, and where the last extension should be attached to the modular core last are entirely determined by the system based on the user's information. It is also contemplated that last extensions may be selected from a digital library of different last extension models. These digital last extension models may or may not have pre-manufactured physical counterparts to be readily used in assembling the last. If last extensions are selected from a digital library, it is additionally contemplated that the dimensions of the selected extensions may be adjusted further during the design of the last extension based on the user's information. In some embodiments, it is envisioned that while modular core last components may be pre-manufactured, last extensions may be manufactured on demand based on the design for the personalized modular last and thereafter added to the modular core last during an assembly process.

Last extensions allow a core last to be extended in certain portions of the core last in order to account for variations in a user's foot or footwear needs that the core last on its own may not be able to provide. Such variations may simply include foot dimensions or foot shape unique to a user and may include certain abnormalities or pathologies a user may have. It is also contemplated that gait and/or biomechanics information, as well as contextual information associated with the user, may also implicate different last extension designs.

Last extensions are typically solid add-ons to the core last that effectively increase the dimensions of the core last in certain areas to provide for extra allowance in certain portions of the final footwear product. FIG. 13 illustrates a personalized modular last that has several last extensions 1303, 1304. One of the last extensions, for example, provides extra allowance for a user's stacked toe. It is envisioned that last extensions may be formed from a material having similar properties to the material used for the core last. As last extensions, like the core last, must perform the function of forming and maintaining a specific foundation and shape of a footwear product while the footwear product components are being pieced together, the last extensions should be made of a material with a suitable hardness for maintaining the intended shoe structure during the manufacturing process. As such, last extensions may be formed from various plastics (such as high-density plastic), synthetic polymers, metals (including metal alloys), or wood, for example, or combinations of these materials. Last extensions may, for example, be 3D printed, as will be described in further detail below.

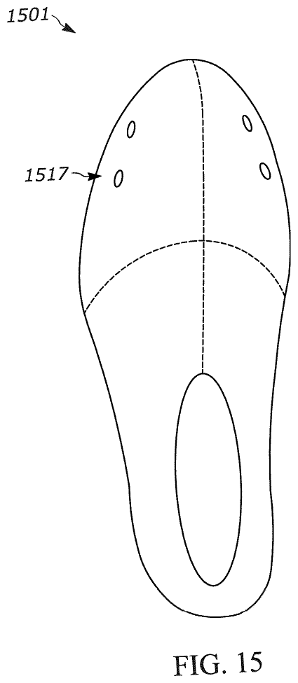

Last extensions may be added to the core last in a variety of ways. In some illustrative embodiments, the last extensions are removably coupled to the modular core last to permit re-use of the modular core last and/or the last extensions in different configurations for different personalized modular lasts, and potentially for different users. This contributes to the efficiency and scalability of the modular last system. In one embodiment, illustrated in FIG. 15, a modular core last 1501 may include apertures 1517 for pins, screws, or snap-on extensions, so that the last extensions may be attached. Other mechanisms for coupling may include but are not limited to other arrangements of apertures for pins, screws, or snap-on extensions, interlocking pieces, velcro attachments, magnets, and removable adhesive. A single last extension may be attached to the core via several discrete fasteners (e.g., several spaced snaps) in case one is insufficient to secure a close fit to the core last. It is further contemplated that the fastening mechanism may be present over the entire interface between the last extension and the core last (e.g., as in the case of adhesive). Further, instead of being removably coupled directly to the modular core last, a last extension can also be removably coupled in the above-mentioned manner to another last extension. This configuration is illustrated in FIG. 13, which shows one last extension 1304 coupled to another last extension 1303.

Determining last extensions includes determining the dimensions of the last extension as well as where on the core last the last extension is to be placed—that is, the precise attachment points or attachment area on the core last. Both determinations are based on the assessment information associated with the user.

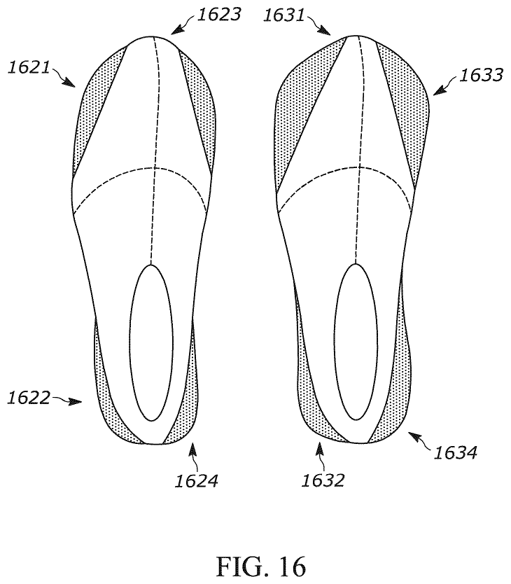

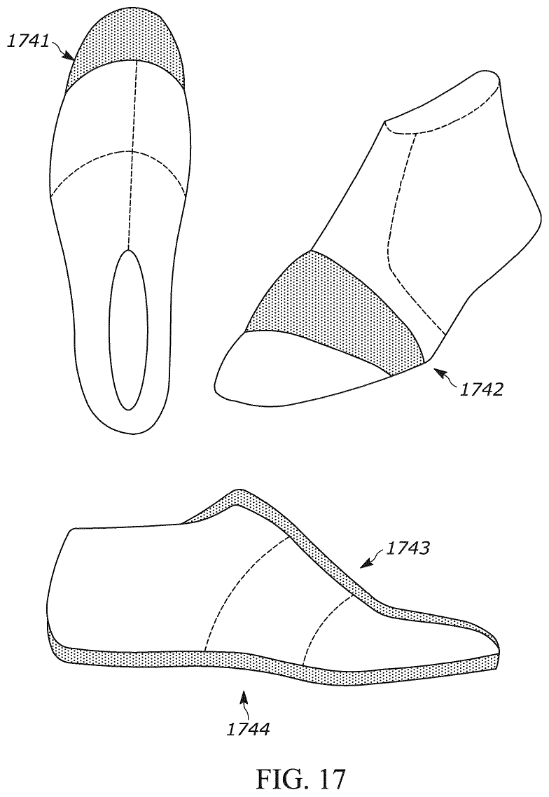

In one approach, it is envisioned that one or more last extensions may be primary extensions. Primary extensions may be understood to be last extensions that are added to specific predetermined regions of a core last. FIGS. 16 and 17 illustrate regions on a core last where a primary extension may be placed. A primary last extension may be removably coupled, for example, to an inner ball region 1623, an outer ball region 1621, a medial heel region 1624, a lateral heel region 1622, a toe tip region 1741, a ball girth region 1742, an instep region 1743, or a bottom region 1744 of the modular core last. These regions may comprise larger or smaller areas depending on the type of shoe last. For example, inner ball region 1623 constitutes a smaller area on one core last compared to an inner ball region 1631 on a different last. It is further contemplated that a primary extension may be determined to extend over a portion of a region or the entirety of a region. Further, in some embodiments the extension may be determined to be contoured or otherwise have an irregular thickness across its area. In this manner, primary extensions may advantageously modify a core last to permit additional personalization of the last and an even closer footwear fit.

Secondary extensions are further contemplated. In one approach, secondary extensions may be understood to be last extensions that may be added anywhere on a personalized modular last. For example, they may be removably attached at any location on the modular core last, or they may be removably attached to a primary extension. FIG. 13, for example, illustrates a personalized modular last 1301 that has a primary extension 1303 attached to the ball girth region of the core last 1302. The last 1301 further includes two secondary extensions 1304. One of the secondary extensions is attached to the primary extension 1303 at a determined attachment point on the primary extension. The other secondary extension is attached at a determined attachment point on the core last. Any number of secondary extensions can be included to personalize the last. Further, in some embodiments secondary extensions are configured to be placed in more precise or localized points on the core last or primary extensions (instead of merely in certain regions of the core last).

In one approach, secondary extensions are determined to be attached to the core last (or to other extensions on the core last) at selected attachment points to correspond to a foot shape abnormality or a pathology of the user. It is contemplated that both the location (e.g., attachment points) and the dimensions of the secondary extension are determined during the design of the personalized modular last based on the user's assessment information. For example, one of the secondary extensions 1304 in FIG. 13 may be sized to correspond to the location of a protruding bunionette. Another secondary extension 1304 may correspond to the location of a stacked toe. For both abnormalities, it is important to include extra allowance in a shoe in those locations to prevent discomfort or worsening of condition. Secondary extensions may provide customization suitable for abnormalities or pathologies such as bunions/bunionettes, toe deformities (e.g., hammer toes, crossed toes, stacked toes), arch variations (flat/collapsed), or bone spurs. However, any number and type of foot shape abnormalities or pathologies can be compensated for in the last via secondary extensions. Furthermore, it is contemplated that other types of information about the user, in addition to foot shape and pathology, such as gait information, biomechanics information, or contextual information, can also inform the determination of secondary extensions.

In some embodiments, secondary extensions, like primary extensions, may be determined to be contoured or otherwise have an irregular thickness across the area of the extension based on the user's needs.

The step of determining one or more last extensions 215 based on the assessment information associated with a user may leverage one or more algorithms to make the determination. One or more machine learning algorithms may be used which may have been trained to use a wide variety of data from a user, including any assessment information, personal attributes, or footwear attributes associated with a user, to determine, for example, which areas of the core last should be extended, the dimensions of the extensions, and the configuration of the extensions (e.g., an extension on top of another extension). Such algorithms include but are not limited to neural networks (e.g., CNN, R-CNN, YOLO), Statistical Shape Analysis (SSA), Support Vector Machine (SVM), Principal Component Analysis (PCA), Bayesian networks, decision trees, nearest neighbor searching, and more.

It will be appreciated that the above design process may be separately performed for both a right foot and a left foot of a user. This is particularly the case if the information provided for each foot is different. As such, two designs for two personalized modular lasts, one for each foot, may be generated for a user. As asymmetry between a right and left foot is quite common, designing different personalized modular lasts for each foot advantageously can provide for proper fit even in the case of asymmetry. Personalized modular last designs for a user's right foot and left foot may end up utilizing different modular core last components (for example, if one foot is longer than another, or if a user suffers asymmetric edema), different primary extensions (for example, if one foot has a narrower heel), or different secondary extensions (for example, if one foot has a bunion).

### Designing Personalized Shoe Components

The present disclosure further includes designing personalized shoe components. An exemplary method, shown in FIG. 1, includes providing a personalized footwear product or design thereof by designing both a personalized modular last and one or more personalized shoe components. However, it is contemplated that the personalized shoe components described herein may be designed for use in a personalized footwear product without designing or using a personalized modular last.

According to method 100, after the design for the personalized modular last for a user is complete, a further step 120 includes designing one or more personalized shoe components. A design program, such as the personalized shoe component design program 390 of system 300, may be used to perform this function. Such a program may be a computer aided design (CAD) program which may generate 2D or 3D models of the designs, however other known forms of digital drawings and design programs known in the art are also contemplated herein.

Generally, a design for a shoe component may include determining the shape, dimensions, and material of the shoe component based on the design for the personalized modular last, the assessment information associated with the user, or both. Personalized shoe components that may be designed in this step are not particularly limited. They may include any component of a footwear product that has any bearing on meeting a user's footwear needs or preferences. By one approach, a complete design for an entire shoe is generated, and, as such, all the components of a shoe are selected. By other approaches, designs for some shoe components, but not all the components of a shoe, are generated.

The design for the personalized modular last provides an advantageous input into shoe component design. As the last design has already, to a large extent, captured and accounted for the subtleties of a user's foot shape, morphology, pathologies, and other footwear needs therein, it is appropriate that the shoe components use the last design as a starting point. For instance, the dimensions of a personalized modular last may prescribe an upper or an outsole with certain dimensions. As another example, a last extension allowing extra space for a bunion may prescribe an insole that is extended correspondingly.

Inputting the last design into the designs for shoe components may be additionally valuable for permitting the possibility of a compensating or synergistic effect between the personalized modular last design and the shoe component designs. For example, if a design for a last does not precisely match a user's foot in one region, the material or thickness of a shoe component (for example, an insole) may be adjusted to permit a better fit.

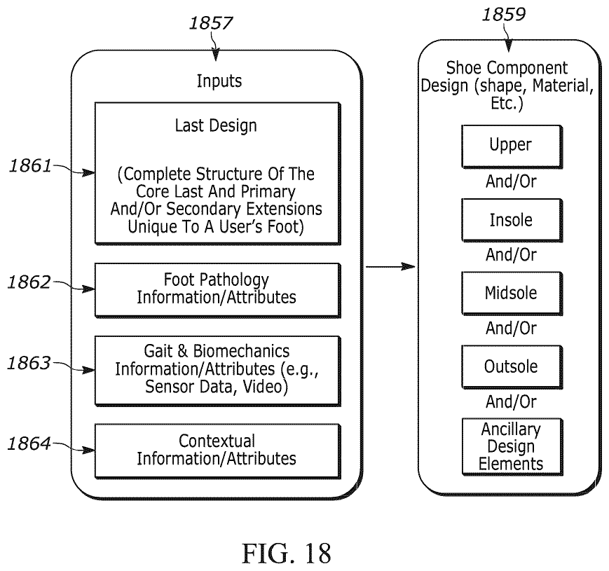

While the last design may provide an integral guide for the design of shoe components, other assessment information associated with the user may also be incorporated into the design to provide a further extent of personalization. As illustrated in FIG. 18, inputs into shoe component design include, e.g., the last design 1861, and may also include foot pathology information or attributes 1862, gait and biomechanics information or attributes 1863, and contextual information or attributes, as described hereinabove. Further included may be foot shape information or attributes not included in the last design.

Exemplary shoe components that may be determined in this step include an upper component, an insole component, a midsole component, and an outsole component, as illustrated in FIG. 18. The shapes and dimensions of these components may depend heavily on the determined dimensions for the personalized modular last. The materials determined for these components, however, may be determined by other assessment information or attributes associated with the user. For example, if a foot pathology attribute associated with the user is a bunion (hallux valgus), tailor's bunion, or other digital deformity, a material for an upper may be selected that has a high elasticity. In another example, if a foot pathology attribute associated with the user is hyperhydrosis, materials with high breathability may be selected for one or more shoe components.

Ancillary design elements may also be determined to be included depending on the needs or preferences of the user, which include but are not limited to laces, straps, air bubbles, lights, traction materials, extra padding, outsole thread pattern, specific textures, foxing, eyestays/eyelets, seams/stitching, collar padding, toe tip reinforcement, added toe spring, vamp lining, printed and/or embossed designs, or extra panels (such as mesh panels).

Algorithms may be leveraged to determine the designs for the shoe components based on the design for the personalized modular last and/or other assessment information associated with the user. One or more machine learning algorithms may be used which may have been trained to use a wide variety of data from a user, including any assessment information, personal attributes, or footwear attributes associated with a user, as well as last design data, to determine ideal shoe component designs for the user. Such algorithms include but are not limited to neural networks (e.g., CNN, R-CNN, YOLO), Statistical Shape Analysis (SSA), Support Vector Machine (SVM), Principal Component Analysis (PCA), Bayesian networks, decision trees, nearest neighbor searching, and more.

In one approach, analogous to the above-described method of determining a modular core last, one or more algorithms use measurements for the last design and other assessment information to identify a closest matching pattern for a type of shoe component from a digital library 392 comprising a range of patterns of different shapes or dimensions for that type of shoe component. For example, to generate a design for a personalized upper component, an algorithm may be used to identify a closest matching upper pattern from an Upper Patterns library. Likewise, a closest matching midsole pattern may be identified from a Midsole Library, a best fitting insole may be identified from an Insole Library, a best fitting outsole may be identified from an Outsole Library, and a best fitting ancillary design element may be identified from an Ancillary Design Element library. In such an approach, digital patterns available to be selected in the digital library 392 may have pre-manufactured physical counterparts that are ready to be incorporated into a footwear product (for example, at a manufacturing facility). However, it is also contemplated that the availability of a digital pattern to be selected from the digital library 392 is not contingent on the availability of pre-manufactured physical counterparts to the digital patterns.

In another approach, one or more algorithms may generate an entirely new pattern for a shoe component based on the last design and/or other assessment information associated with the user.

In a middle approach, an algorithm may be used to identify a closest matching pattern for a type of shoe component from a digital library 392 based on the last design and/or other assessment information associated with the user, but then may re-size or otherwise modify those upper patterns based on the last design and/or other assessment information associated with the user. This may be a preferred approach in some embodiments, as it allows shoe components to be customized to match a user more precisely, while also leveraging the value of “tried and true” pre-set patterns by using those patterns as a base for further personalization.

In addition, one or more shoe components may be designed in part or in whole by a human designer, based on the last design and/or other assessment information associated with the user.

In some embodiments, an illustrative method or system for designing personalized shoe components includes obtaining assessment information associated with a user (where the assessment information may include at least one foot measurement of the user) and generating a design for one or more personalized shoe components, wherein generating the design for the one or more personalized shoe components includes, for each of the one or more personalized shoe components. By one approach, the method or system also uses an algorithm to select a predesigned or preset shoe component design (or “pattern”) from a library of preset shoe component designs based on a design for a personalized modular last personalized to the user (such as being based on the user's foot measurements or assessment information) and, optionally, the assessment information associated with the user. In some configurations, these teachings optionally use an algorithm to resize the selected preset shoe component design(s) based on the design for the personalized modular last. Further, the methods and systems may select a material for the personalized shoe component based on the specific design of the personalized modular last and/or the assessment information associated with the user.

The personalized shoe components that are designed may include at least one of an upper component, an insole component, a midsole component, and an outsole component, or any combinations of these components. For example, at least two might be designed (a midsole and an outsole), at least three might be designed (an upper, an insole, and an outsole), or all four might be designed.

Such a method or system may further include determining at least one ancillary design element to be added to the design, wherein the at least one ancillary design element includes one or more of laces, straps, air bubbles, lights, traction materials, extra padding, outsole thread pattern, specific textures, foxing, eyestays, eyelets, seams, stitching, collar padding, toe tip reinforcement, added toe spring, vamp lining, printed and/or embossed designs, and extra panels.

In one approach, an upper component in particular may be designed, which includes obtaining assessment information associated with a user, the assessment information comprising at least one foot measurement of the user and generating a design for a personalized upper component. In some approaches, generating the design for the personalized upper component includes using an algorithm to select a preset upper design from a library of preset upper designs based on a design for a personalized modular last personalized to the user (such as being based on the foot measurements of the user or the user's assessment information), and, optionally, the assessment information associated with the user. Further, to get additional customization to a user, an algorithm may resize the selected preset upper design based on a the design for the personalized modular last and, optionally, the assessment information associated with the user. In some embodiment, the user or design can select a material for the personalized upper component based on the design for the personalized modular last and/or the assessment information associated with the user.

It will be appreciated that a process analogous to the above process could occur for a midsole component, an insole component, or an outsole component.

Further, in some configurations certain input information may be particularly relevant to the design of a specific shoe component. As an example, in one case the design of an upper may be based on the design for the personalized modular last (which incorporates at least the user's foot size and/or shape), and may also be based on foot pathology information. In another case, the designs for an insole, outsole, or midsole may be based on the design for the last, and may also be based on foot pathology information, gait information, and/or biomechanics information. The custom design or inclusion of specific ancillary design elements, in another case, may be based on the design for the last, and also may be based on foot pathology information, gait information, biomechanics information, and/or contextual information associated with the user. For example, information about a user's foot size and shape (from the design for the personalized modular last personalized to the user), coupled with information about the user's gait and information about the user's typical activities (if, for example, the user is a runner), may implicate a design for added padding or certain laces.

The materials selectable for use in the personalized shoe components may be limited by the methods of manufacturing that may be used to manufacture the components. Further, selected materials for use in the personalized shoe components may implicate specific manufacturing methods. Accordingly, determining a material for a personalized shoe component, in some embodiments, is based in part on the relationships between materials and available manufacturing methods.

It is contemplated that a variety of different manufacturing techniques may be used to manufacture the personalized shoe components disclosed herein, including but not limited to additive, subtractive, and/or near net shape manufacturing, including, for example, 3D printing, 3D knitting, die cutting, and laser cutting. Depending on the technique used to manufacture the shoe components, as stated above, different materials for different shoe components may be selected.

For example, laser cutting a shoe component design out of a sheet of material may be a preferred technique for certain components. Certain materials, however, have more or less compatibility with laser cutting. An upper component may cut well if it is made of natural leather or suede at a maximum thickness of ⅛ inch, or if it is made from a fabric (for example, cotton, polyester, Gore-Tex, felt). However, materials containing PVC such as artificial leather or pleather, should be avoided, as such materials emit chlorine gas when they are cut. Plastic coated or impregnated cloth should also be avoided.

As another example, an insole component that is to be laser cut may be made, for instance, from a polyester foam, a polyethylene foam, a polyurethane foam, an EVA foam, or a cork. Foams such as polyester (PES), polyethylene (PE) or polyurethane (PU) are particularly suitable for laser cutting. Contactless processing ensures fast cutting without exerting pressure on the material, while heat from the laser beam seals the edge. Ethylene-vinyl acetate (EVA) foam in particular may be preferred for laser cutting and engraving an insole.

Suitable materials for laser cutting a midsole component include, for example, open cell polyurethane, closed cell EVA, or Gore-Tex, while rubber (comprising no chlorine) and EVA, for example, may be suitable materials for laser cutting an outsole.

While the above-described functions of designing a personalized modular last and designing personalized shoe components were described using assessment information associated with a user as a basis for the designs, it will be appreciated that personal attributes or footwear attributes, as discussed in the foregoing, may also be used as a basis for the designs. For example, an exemplary system for providing or designing a personalized footwear product may include at least one processor communicable with at least one electronic user device, the processor configured to receive assessment information associated with a user from the at least one electronic user device, the assessment information including at least one foot measurement; determine one or more footwear attributes associated with a user based on the assessment information; and generate a design for a personalized modular last, wherein the personalized modular last comprises a modular core last. In some approaches, generating the design for the personalized modular last includes determining a modular core last model by selecting one or more modular core last model components from a library of modular core last model components, based on at least one of the one or more footwear attributes associated with the user. Further, the processor may be further configured to generate a design for at least one personalized shoe component, based on the design for the personalized modular last and/or one or more of the one or more footwear attributes associated with the user. An exemplary method comprising the above-mentioned functions is illustrated in FIG. 12.

Determining footwear attributes may be advantageous in designing the personalized footwear, as footwear attributes provide a nexus between the design of the footwear and the personal information or attributes related to a user's foot shape, pathologies, gait and biomechanics, or other contextual information. For example, footwear attributes may have the following relationships with foot shape, pathology, gait and biomechanics, and contextual information:

TABLE 1

| Factor | Description |
|---|---|
| Footwear Attribute | May consider the influence of 3D foot shape |
| Relationship with | by shoe material properties and how footwear |
| Foot Shape Attributes | attributes interact with foot surface |
| | morphologies |
| Footwear Attribute | May consider interaction of footwear material |
| Relationship with | with the foot pathologies, including |
| Foot Pathology | predicting long term effect of a material, shoe |
| Attributes | construction, and design on specific |
| | pathology |
| Footwear Attribute | May consider the effect of material and |
| Relationship with | construction on a user's walking pattern, foot |
| Gait and Biomechanics | posture, foot plantar pressure, and activity |
| Attributes | level |
| Footwear Attribute | May consider footwear category, style, and |
| Relationship with | use case |
| Contextual Attributes | |

Determining footwear attributes for users may, accordingly, may optimize designs for personalized modular lasts and/or personalized shoe components.

### Manufacturing a Personalized Footwear Product

It is further contemplated that a personalized footwear product may be manufactured based on the personalized modular last and/or the design for the personalized shoe components. In illustrative embodiments, a personalized footwear product is manufactured based on both the design for the personalized modular last and the design for the personalized shoe components.

FIG. 4 illustrates a method 400 for providing a personalized footwear product that includes manufacturing of the product. After the designs for the personalized modular last and/or the one or more personalized shoe components have been completed, an optional further step 425 includes sending the designs to a manufacturing execution system 360. A manufacturing execution system 360, as used herein, may control one or more operational parameters for a footwear manufacturing process, and may include one or more manufacturing devices that are configured to perform a function in manufacturing a footwear product. For example, a manufacturing execution system may include the combinations of core last and extensions, an algorithm for instructing a machine to perform additive manufacturing, an algorithm for instructing a machine to perform subtractive manufacturing on a material using specific spatial coordinates, and/or a set of instructions to assembling products. As FIG. 3 indicates, the manufacturing execution system 360 may also include a store of pre-manufactured modular last components 369, or other pre-manufactured components (such as last extensions or shoe components) that may be readily used (“on demand”) to manufacture the personalized footwear product disclosed herein. These components may be pre-manufactured on site or come from a supplier.

The generation of the footwear product through manufacturing may be initiated via the personalized footwear design and/or production engine 336, or by some other means. For example, the personalized footwear design and/or production engine 336 may send a signal to one or more manufacturing devices (e.g., 361, 363, 365, 367) in the manufacturing execution system 360 to control device operation. The signal may be a program consisting of a code to run the machine or instruct the machine to perform a specific manufacturing task.

Generally, as indicated in FIG. 4, manufacturing a personalized footwear product as disclosed herein includes the steps of 430 providing the personalized modular last on the basis of the design for the personalized modular last, 435 manufacturing the one or more personalized shoe components on the basis of the design for the one or more personalized shoe components, and 440 using the personalized modular last to manufacture the personalized footwear product, which includes incorporating the one or more personalized shoe components into the product. In addition, in some embodiments a personalized footwear product may be utilize a personalized modular last in being manufacture but may not include any personalized shoe components. Further, in some embodiments one or more personalized shoe components may be manufactured that constitute the final personalized footwear product. For example, a user may require solely one or more personalized shoe components for use in a shoe that is already owned, and, as such, the personalized shoe component is not incorporated into a newly manufactured shoe. Additionally, it is contemplated that in some cases during manufacturing of a footwear product the personalized shoe components may be incorporated into the footwear product without using a personalized modular last.

The manner in which the personalized modular last is manufactured and/or assembled depends to a certain extent on the complexity of the design for the personalized modular last. It is envisioned that in simpler designs, such as when a design indicates a binary core last with two specified modular core last components, the two modular core last components have been pre-manufactured 369 and are present, and merely need to be identified and assembled (that is, removably coupled together, in any one of the manners described above).

Further, a core last and/or extensions catalog and a numbering system may be developed for assisting in the manufacturing and assembly of the last and shoe. The catalog may, for instance, be alphabetical and/or numerical. For example, a core last component may be designated 8D while two last extensions may be designated A108 and B107. Any number of cataloging systems may be used so that the components may be catalogued and identified. Assembly of core last components and extensions may be performed by a machine or by hand.

If the design for the personalized modular last indicates that one or more last extensions are required, pre-manufactured last extensions may be used (for example, already manufactured to use in a previous last) and removably coupled to the modular core last at the attachment points indicated by the design. By such an approach, the pre-manufactured last extensions may not perfectly match the last extensions determined by the design. As such, it may be particularly advantageous to manufacture the required last extensions on demand, and, subsequently, removably couple the manufactured last extensions to the modular core last to provide the personalized last. The last extensions may be made via any suitable manufacturing device, including additive manufacturing devices (such as 3D printing machines), subtractive manufacturing devices (such as a laser cutter and/or CNC machine, etc), and/or near net shape manufacturing devices (such as stamping, etc).

The one or more personalized shoe components, as mentioned above, may be pre-manufactured, as mass produced or customized manufactured, or may need to be manufactured on demand, depending on the design approach. As explained, if the design for the personalized shoe components determines a shoe component by selecting a preset pattern from a digital library 392 that is the closest match for a user, such a pattern may be pre-manufactured and available for use in manufacturing and/or assembling the footwear product. However, if the design process determines a shoe component by re-sizing a preset pattern to provide a better fit to a user, or by generating a pattern from scratch, the shoe components may need to be manufactured on demand.

As discussed above, a variety of manufacturing techniques may be used to manufacture the shoe components, which may depend on the type of shoe component and/or the type of material used. In some approaches, for example, a 3D printing machine may be instructed to produce a portion of a shoe, such as, e.g., the sole, insole, and/or footbed and a 3D knitting machine may be instructed to complete other portions of the shoe, such as, e.g., the upper and tongue. Furthermore, subsequently a 3D printing machine may be instructed to produce additional portions of the shoe, such as, e.g., a toe cap. As described above, laser cutting may be a preferable manufacturing technique for many shoe components.

Upon provision or manufacturing of the personalized modular last and the shoe components, the personalized modular last is used to manufacture the footwear product and the shoe components are incorporated into the footwear product using lasting and shoemaking techniques known in the art. For example, when a shoe is in production, a stitched pattern may be stretched over the last to create the shape of the shoe (called “lasting”). There are several different lasting techniques that may be employed to pull the patterns into shape. These include force lasting, board lasting, string lasting, toe lasting, heel lasting, hand lasting, and machine lasting. Once the pattern is tight to the last, the outsole can be attached. The last holds the soft upper in place, whether the sole is sewn on or cement-bonded.

In this manner, a personalized footwear product, closely aligned with a user's personal footwear needs, is provided.

It is further contemplated that the one or more modular core last components and the one or more last extensions are decouple-able after use in manufacturing the personalized footwear, and stored and re-usable to assemble one or more different personalized modular lasts to provide different personalized footwear products for the same user or for one or more different users.

In some embodiments, the system 300, for example, via the personalized footwear design and/or production engine 336, may also be communicable with a shipping system. In one embodiment, the engine 336 may send a signal to the shipping system to automatically place an order for, manufacture, and/or ship one or more manufactured personalized footwear products to a particular user and/or individual. In one example, the footwear products could be shipped directly to the user. In another example, the footwear products could be shipped to a store or nearby location for pick-up. In another approach, the engine 336 may first identify recommended personalized footwear designs for a particular user and/or individual. In one approach, the user may select one or more of the recommended personalized footwear designs to place an order and have the products manufactured and shipped. In some embodiments, the engine 336 may both control the manufacturing of a footwear product for a particular user and automatically ship the product to the user. In this manner, the engine 336 may customize orders for a particular user. It is also contemplated that, after shipping or otherwise providing one or more custom footwear products to a user, the system 300 may also receive feedback on the footwear product(s). For example, the system 300 may transmit a questionnaire or survey to the user to receive information, for example, on the comfort, fit, aesthetics, or other experiences with the footwear product and for example, whether the user plans to keep the footwear product or would like certain adjustments to the product.

For example, in one embodiment a medical practitioner may place an order for a patient after using an electronic user device to input assessment information about the patient. In such a case, the footwear products may be designed and manufactured, and may be shipped directly to the patient or to the office of the medical practitioner. After the patient has worn the footwear product for some time, the medical practitioner may follow up with the patient and ask for feedback about the footwear product. The medical practitioner may use the feedback to place a subsequent order for an adjusted footwear product for the patient, if necessary or desired.

### Post-Wear Feedback and Analysis

In some embodiments, the system 300 may be operable to obtain feedback on the footwear product directly and/or indirectly from the user to make future adjustments to the design of the footwear product in order to provide a constantly improved cycle of new footwear products for a user. The feedback from a previously worn footwear can change, for example, the design, construction, and/or material of the new footwear. The feedback may be provided directly by the user, or may be provided by other parties. In the former case, the user may provide feedback by providing information regarding the effects of the previously worn footwear product on the user's foot shape, pathologies, gait, preferences, etc, via any of the manners and methods described herein. In the latter case, feedback regarding the previously worn footwear product may be determined from a scientist or researcher, a medical practitioner, a designer, a manufacturer, etc., and can include, for example, extracting and analyzing sensor data, conducting post-wear analyses of the footwear construction and material, and considering other aspects of the interaction between a user's foot and the footwear product. These measures of obtaining feedback regarding a previously worn footwear product are described below.

By one approach, the methods herein may be leveraged to produce personalized footwear for a particular individual that is designed in light of the particular individual's foot pathologies, gait, biomechanics, and other usage-related aspects. Furthermore, the personalized footwear is likely to evolve over an individual's lifetime and the analysis of footwear worn by the particular individual, along with potentially analyzing updated scans and other sensed information, may be leveraged to adjust the footwear recommendation or footwear manufactured, to account for changes. Further, receiving feedback on previously designed or manufactured footwear creates an iterative process that results in a particular individual receiving updated designs for footwear and footwear products over time. This method is in contrast to the standard mass manufacture of footwear that provides limited sizing options and that is built on a limited understanding of consumer needs, resulting in poor fit and thereby poor health (in light of the documented health consequences of the wearing of improperly sized footwear).

It is further contemplated that footwear usage data and/or post-wear data of a previously worn footwear by a user, including a previously worn personalized footwear product made in accord with the systems and methods disclosed herein, may be included in the assessment information associated with the user provided to design and/or manufacture a personalized footwear product.

For example, one illustrative system may include at least one processor communicable with at least one electronic user device, the processor configured to: obtain assessment information associated with a user, the assessment information comprising feedback regarding a previously worn footwear product, generate an updated design for a personalized footwear product, the updated design for a personalized footwear product including a design for a personalized modular last and a design for at least one personalized shoe component, wherein the design for the personalized modular last includes a modular core last model which is determined by selecting one or more modular core last model components from a library of modular core last model components, based at least in part on the feedback regarding a previously worn footwear product, wherein the design for the at least one personalized shoe component is based at least in part on the design for the personalized modular last and/or the feedback regarding a previously worn footwear product.

A further illustrative system includes at least one processor communicable with at least one electronic user device, the processor configured to: obtain assessment information associated with a user, the assessment information comprising at least one foot measurement of the user, generate a design for a personalized modular last, wherein the personalized modular last comprises a modular core last, wherein generating the design for the personalized modular last includes determining a modular core last model by selecting one or more modular core last model components from a library of modular core last model components, based at least in part on the at least one foot measurement of the user, the processor further configured to generate a design for at least one personalized shoe component, based on the design for the personalized modular last and, optionally, the assessment information associated with the user; the processor further configured to obtain feedback regarding a previously worn footwear product that was manufactured based on the design for the personalized modular last and the design for the personalized shoe component; the processor further configured to generate an updated design for the personalized modular last and/or an updated design for the at least one personalized shoe component based at least in part on the feedback regarding a previously worn footwear product.

The feedback regarding a previously worn footwear product can include updated assessment information such as foot shape information, gait information, biomechanics information, pathology information, and/or contextual information.

Further, in use, the systems and methods herein may update a user profile associated with a user based on the feedback regarding the previously worn footwear product. The user profile may store information associated with a user such as feedback regarding the previously worn footwear product, as well as one or more previous designs for previously personalized footwear products, and may be communicable with the design software programs to update the designs based on the feedback.

Receiving feedback regarding a previously worn footwear product may permit the design for the personalized footwear product to be updated in different ways. For example, the feedback may result in the updated design including at least one change of construction, at least one change of material, or a combination of least one change of construction and at least one change of material for the personalized footwear product compared to the previously worn footwear product (or the previous footwear design the previously worn footwear product is based on). The design for the personalized last may be updated (for example, one or more last extensions may be added to the last), the design for one or several different shoe components may be updated, or both the last and shoe component designs may be updated. In some cases, the system may determine that no updates to the design are needed based on the post-wear feedback.

In some embodiments, the feedback includes an analysis of previously worn footwear to determine a degree of wear and tear patterns, among other aspects. The shoes can be analyzed either intact or as individual components to determine the qualitative and quantitative degradation of the structure and material of the shoe after wear. The structural changes may be determined, for example, by comparing the previously worn shoe or a component thereof with the original shoe shape, foot shape, gait, biomechanics, and/or contextual information associated with the user. For example, the structure of an upper component may be deformed compared to the original shape. The material changes may be determined, for instance, by assessing in light of the original shoe shape, foot shape, gait, biomechanics, and/or contextual information associated with the user. For example, the material of an insole or midsole may be compressed, fatigued, cracked, uneven shaped, or torn. The changes in the structure and material of the footwear product may be quantified by use of structural mechanical tests, including but not limited to, torsional flexibility, tensile strength, bending, stretching, pull-out, creep, and relaxation tests.

In use, these teachings permit a user to wear initial personalized footwear formed on modular lasts (such as footwear manufactured via the processes described herein) and return the initial personalized footwear for post-wear processing or analysis to provide an updated personalized footwear. For example, if a portion of the shoe has been stretched or deformed, the systems described herein may update a portion of the updated personalized footwear (such as a shoe component) to have a particular elastic or flexible material at the location and/or may form the updated personalized footwear on a modular last that has an added last extension (e.g., primary or secondary last extension) to the stretched or deformed area.

In one illustrative embodiment, discussed further below, the feedback may include footwear condition data. The systems and methods may employ one or more sensors in footwear to obtain footwear condition data on, for example, temperature, humidity, acceleration, pressure, alignment, posture/tilt, and/or other footwear attributes of the footwear during footwear usage to provide updated designs based on how a particular user wears the footwear. By one approach, the measured data may be compared to other measured attributes from a database for comparison purposes.

In use, the sensed data may be combined with other data provided and/or gathered about the user. For example, the methods herein may leverage both two-dimensional measurements, three-dimensional measurements, and dynamic measurements, such as, for example, foot measurements, foot scans, still image, and/or video data obtained by a camera or sensor external to the footwear, and/or shoe-mounted sensors configured to obtain data on the microclimate within the footwear as described below. By leveraging some or all of this data, the systems and methods herein assist with reducing the major shoe pain points of users including heel slippage, arch support, and toe box fitment. For example, while ball width or girth, heel diagonal, and arch height may be statically measured, dynamic analysis of the footwear in use permits measurement of the sole flexion point, the plantar or dorsiflexion point, and the level of arch flexibility or rigidity, and accommodating for these factors greatly improves wearer comfort levels.

As described below, the methods and systems are configured to measure humidity and temperature (to ascertain the microclimate within the footwear being worn) and acceleration and pressure (to ascertain a user's alignment and gait), which may then be accounted for in the personalized footwear design and/or manufacture.

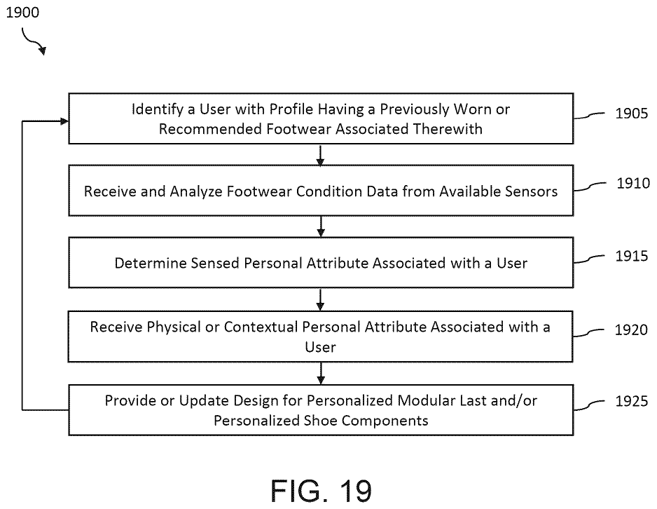

FIG. 19 illustrates an exemplary method 1900 of providing or updating a personalized footwear design based at least in part on analyzing footwear condition data to determine a sensed personal attribute associated with a user. In some embodiments, one or more of the steps of method 1900 may be executed in conjunction with the personal attribute analysis module 328 described with reference to FIG. 3. In some approaches, both physical and/or contextual information, may be associated with the user in a database, such as the databases described in further detail below or those described above with respect to FIG. 3.

The method 1900 includes identifying 1905 a user profile having previously worn footwear associated with the user and receiving and analyzing 1910 footwear condition data associated with the user from one or more sensors. For example, the sensors may include a pressure sensor, accelerometer, a gyroscope, a humidity sensor, and/or a temperature sensor, among others.

The method 1900 also includes determining 1915 the sensed personal attribute to associate with the user profile based on the footwear condition data from one or more sensors.

In step 1920, the method 1900 includes receiving personal attribute data or assessment information, which may include both physical and contextual data, to associate with the user profile. The method also includes providing or updating 1925 the designs for the personalized footwear product, which may include the personalized modular last and any personalized shoe components, at least in part, based on the footwear condition data, the sensed personal attribute, and/or a physical (e.g., foot shape, pathology, gait, biomechanics) or contextual personal attribute or assessment information. The updates to the design for the personalized footwear product may include, for example, updates to the selected personalized modular last (its selected modular core components, its selected last extensions and the locations of the extensions), and updates to the personalized shoe components (e.g., the type or number of components included, the dimensions of the components, the materials of the components).

The method 1900 may repeat after providing or updating 1925 the personalized footwear design back to identifying 1905 a user profile having previously worn footwear associated therewith. In this manner, the method may be iterative and continuously useful to users.

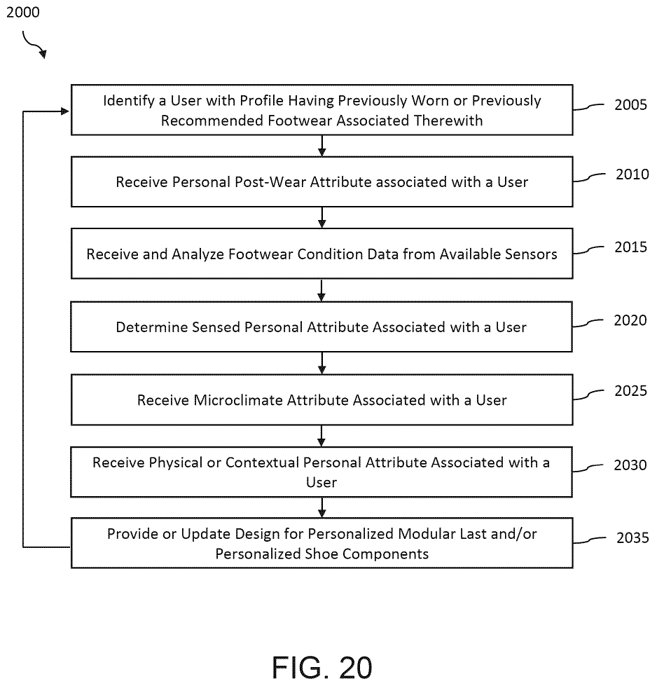

FIG. 20 illustrates an exemplary method 2000 of providing or updating a personalized footwear design in accordance with some embodiments. In some embodiments, one or more of the steps of method 2000 may be executed in conjunction with the personal attribute analysis module 328 described with reference to FIG. 3. In some approaches, both physical and/or contextual information, may be associated with the user in a database, such as the databases described in further detail below.

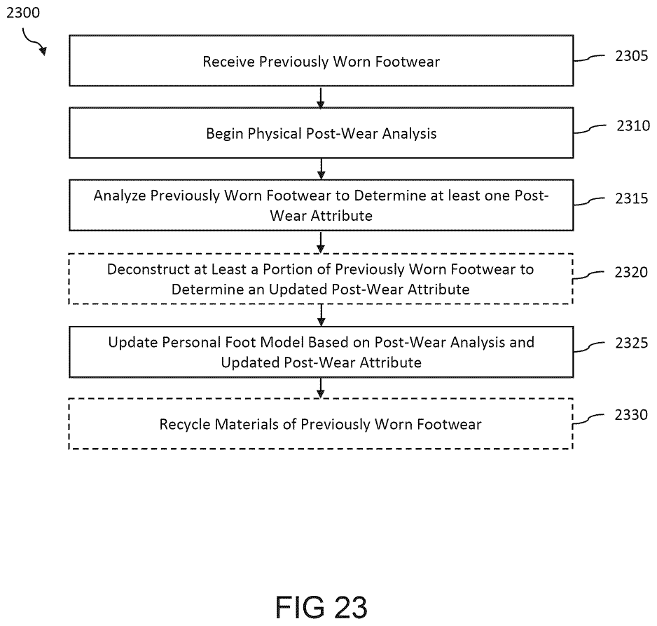

The method 2000 includes identifying 2005 a user profile having previously worn footwear associated with the user. In some embodiments, the method includes receiving 2010 a personal post-wear attribute associated with the user profile, where the personal post-wear analysis is used to determine the personal post-wear attribute, such as the analysis described in further detail below with reference to FIG. 23.

The method 2000 also typically includes receiving and analyzing 2015 footwear condition data associated with the user profile from one or more sensors. For example, the sensors may include a pressure sensor, accelerometer, a gyroscope, a humidity sensor, a temperature sensor, or any other available sensor. The method 2000 also includes determining 2020 the sensed personal attribute to associate with the user profile based on the footwear condition data.

The method 2000 also includes receiving 2025 a microclimate attribute associated with the user profile. The microclimate attribute is determined by a microclimate analysis which analyzes data at least in part from at least one of a humidity sensor or a temperature sensor. The analysis determines aspects of temperature and/or humidity within the footwear with sensors placed in certain portions throughout the footwear to determine a localized microclimate in a certain portion of the footwear. The microclimate data may be used to update the footwear design, or the user profile accordingly through the use of a processor or an electronic user device. In addition to the microclimate data sensed within the shoe, in some embodiments, the methods and systems described herein may solicit user preference or assessment data regarding how the user felt about or responded to the various sensed microclimate levels within the footwear. The microclimate, at least in part, may be determined from data from the humidity and/or temperature sensor and may be used to determine a user's preferred microclimate for comfort, but may also have a predefined database to determine a footwear recommendation based on other physical or contextual personal attributes discussed above, include but not limited to, geographic location such as altitude, ambient humidity of the surrounding area, or time of year.

In some configurations, the method 2000 also includes receiving 2030 personal attribute data or personal assessment information, both physical and/or contextual, to associate with the user profile. The method also includes providing or updating 2035 the designs for the personalized footwear product, which may include the personalized modular last and any personalized shoe components, at least in part, based on the footwear condition data, the sensed personal attribute, and/or the physical (e.g., foot shape, pathology, gait, biomechanics) or contextual personal attribute or assessment information. The updates to the design for the personalized footwear product may include, for example, updates to the selected personalized modular last (its selected modular core components, its selected last extensions and/or the locations of the extensions), and updates to the personalized shoe components (e.g., the type or number of components included, the dimensions of the components, the materials of the components).

The method 2000 may repeat after providing or updating 2035 the personalized footwear design back to identifying 2005 a user profile having previously footwear associated therewith. Indeed, while the initial step 2035 may provide a personalized footwear design, subsequent analysis may update the previous design.

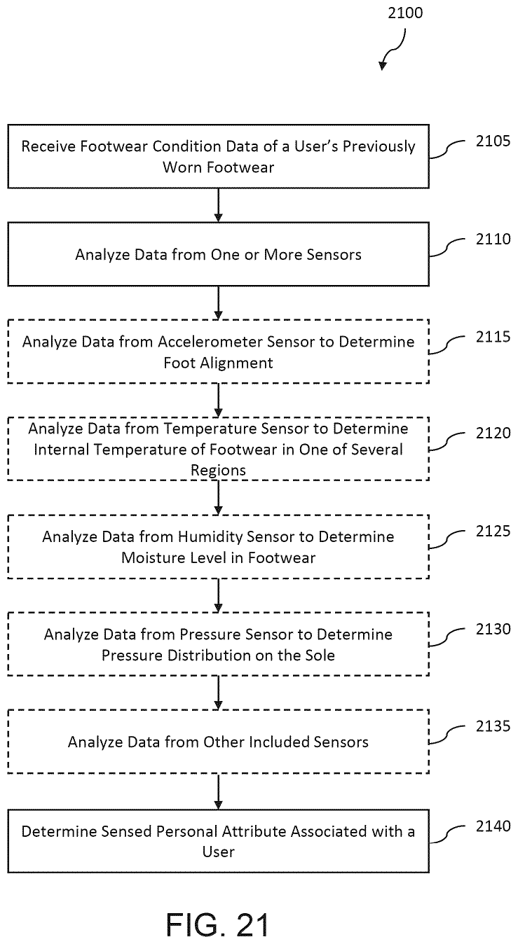

FIG. 21 illustrates an exemplary method 2100 of determining a sensed personal attribute in accordance with some embodiments. The method 2100 includes receiving 2105 footwear condition data of a user's previously worn footwear. The footwear having been worn for a duration of time. In some configurations, the duration of time is several months. In other configurations, the duration of time is about 6 months, but shorter and longer durations are contemplated herein. The method 2100 further includes analyzing 2110 data from one or more sensors.

Analyzing 2110 data from the one or more sensors optionally includes analyzing 2115 data from an accelerometer. By one approach, the accelerometer assists with determination of foot alignment in the footwear. Furthermore, the accelerometer may have other uses such as identifying and monitoring foot strike and foot alignment patterns throughout an individual's gait cycle when walking, running, and performing other athletic and physical activities and monitoring mobility of patients managing medical conditions such as those recovering from a stroke. The present disclosure contemplates both a single accelerometer and several accelerometers strategically placed within the footwear to determine the acceleration or orientation in several regions of the footwear.

Analyzing 2110 data from one or more sensors optionally includes analyzing 2120 data from a temperature sensor. The temperature sensor may determine internal temperature inside the footwear or around the foot, but the temperature sensor may have other uses such as measuring localized temperature variations throughout different regions of the foot which may be indications of tissue injury. The present disclosure contemplates both a single temperature sensor and several temperature sensors strategically placed within the footwear to determine the temperature in several regions of the footwear, and to aid in determining the microclimate attribute described above in FIG. 20.

Analyzing 2110 data from one or more sensors optionally includes analyzing 2125 data from a humidity sensor, but the humidity sensor may have other uses such as providing humidity values which may indicate environments of microbial growth that influence spread of potential bacterial or fungal infections. The humidity sensor may determine moisture level of the interior of the footwear. The present disclosure contemplates both a single humidity sensor and several humidity sensors strategically placed within the footwear to determine the humidity in several regions of the footwear, and to aid in determining the microclimate attribute described above in FIG. 20. It is further contemplated that the humidity sensor may track the humidity of the outside surroundings in conjunction with and/or separate from the interior of the footwear.

Analyzing 2110 data from one or more sensors optionally includes analyzing 2130 data from a pressure sensor. The pressure sensor may determine regions of high pressure on the sole (and/or other areas) of the footwear, but the pressure sensor may have other uses such as identification of foot strike patterns and foot alignment during an individual's gait cycle. The present disclosure contemplates both a single pressure sensor and several pressure sensors strategically placed within the footwear. The present disclosure also contemplates one pressure sensor covering substantially all of the sole of the footwear.

The present disclosure further contemplates other sensors to track other footwear aspects, such as a gyroscope. The gyroscope may, by one approach, assist with determination of foot alignment in the footwear. Furthermore, the gyroscope may have other uses such as identifying and monitoring foot strike and foot alignment patterns throughout an individual's gait cycle when walking, running, and performing other athletic and physical activities and monitoring mobility of patients managing medical conditions such as those recovering from a stroke. The present disclosure contemplates both a single gyroscope and several gyroscopes strategically placed within the footwear to determine the acceleration or orientation in several regions of the footwear.

The method 2100 further includes determining 2140 a sensed personal attributed associate with the user and may update the user profile through the use of a processor and/or an electronic user device. This method 2100 may be associated with method 1900, specifically determining 1915 a sensed personal attribute. This method 2100 may be associated with method 2000, specifically determining 2020 a sensed personal attribute.

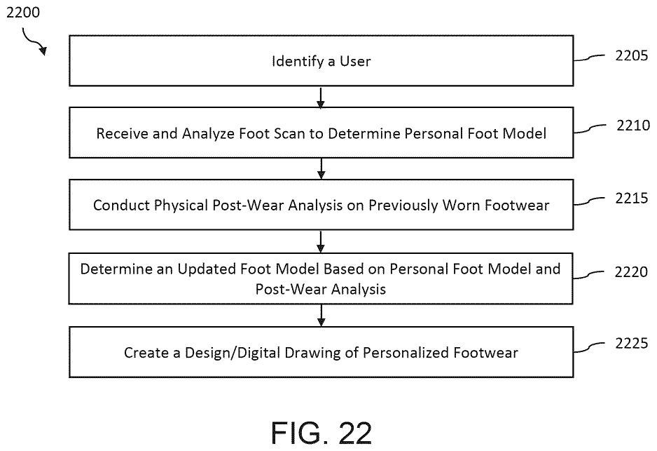

FIG. 22 illustrates an exemplary method 2200 of creating a digital drawing, such as, e.g., a computer-aided design (CAD) drawing, of a personalized footwear design in accordance with some embodiments. The method 2200 typically includes identifying 2205 a user. This user may be a user with previously designed footwear as disclosed herein, a new user, a user with a user profile, or any other user. In one illustrative approach, the method 2200 includes receiving and analyzing 2210 a foot scan to determine a personal foot model. The foot scan may come from an electronic device, such as a phone, a computer, a scanning module, or any other imaging device that contains an image sensor (e.g., a camera). Foot scans may also come from a commercially available foot scanner and may use other imaging technologies, such as laser, non-ionizing radiation, and/or low ionizing radiation.

The method 2200 also includes conducting 2215 a physical post-wear analysis on previously worn footwear. The previously worn footwear may be previously designed personalized footwear as disclosed herein, or any other footwear worn for a duration of time. The physical post-wear analysis described in further detail below with reference to FIG. 23.

The method 2200 also includes determining 2220 an updated foot model based on the personal foot model from the foot scan of 2210 and the physical post-wear analysis of step 2215, which may update the user profile accordingly through the use of a processor or an electronic user device.

The method 2200 also includes creating 2225 a digital drawing of a footwear design. The digital drawing of a footwear design may be done by creating a computer aided design (CAD) drawing, however other known forms of digital drawings are contemplated herein. The footwear design may be of the footwear, a portion of the footwear, or a footwear last conforming to the user's foot based on the updated foot model of step 1720, or may update the user profile accordingly through the use of the processor or the electronic user device.

FIG. 23 illustrates an exemplary method 2300 of conducting a physical post-wear analysis. The method includes receiving 2305 previously worn footwear. By some approaches, the previously worn footwear has been worn for over about 6 months however, shorter and longer durations are contemplated herein. The method 2300 also includes beginning 2310 the physical post-wear analysis which includes analyzing 2315 the previously worn footwear to determine at least one post-wear attribute. The method 2300 also optionally includes deconstructing 2320 at least a portion of the previously worn footwear to determine an updated post-wear attribute and may update the user profile accordingly through the use of an electronic user device.

The method 2300 also may include updating 2325 a personal foot model based on at least one of the post-wear attributes and/or the updated post-wear attribute, which may update the user profile accordingly. The method 2300 optionally includes recycling 2330 the materials of the previously worn footwear, either in whole, or in portions if deconstructed. For example, the shoe may be partially (or wholly) deconstructed to conduct a portion of the data analysis, which thereby breaks down the shoes into constituent pieces that render themselves better suited for being recycled. The ability to recycle their footwear and improve their next pair of shoes is attractive to many consumers.

In some configurations, the physical post-wear analysis of previously worn footwear helps determine certain aspects of the fit and/or usage of the previously worn footwear. The post-wear analysis looks, for example, at the wear, or wear pattern, of the sole of the previously worn footwear, for example, the wear on the heel region of the previously worn footwear, material degradation, and other indications on the sole, insole, midsole, or other portions of the footwear to determine pressure points where high pressure and wear has been put on the previously worn footwear. The physical post-wear analysis may include deconstructing at least a portion of the previously worn footwear. In addition, the methods described herein may further include recycling all or portions of the previously worn footwear.

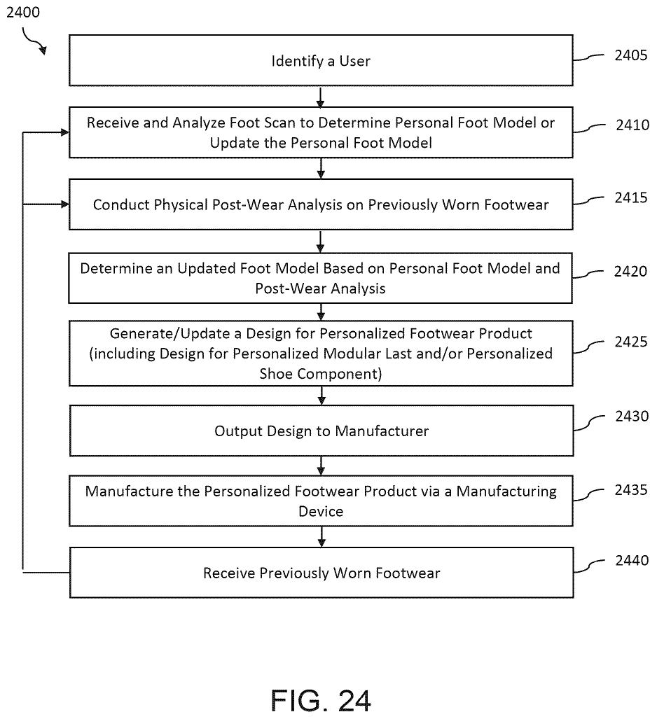

FIG. 24 illustrates an exemplary method 2400 of manufacturing, repeating, and updating a footwear design. The method 2400 includes identifying 2405 a user. The method 2400 also includes receiving and analyzing 2410 a foot scan of the user's foot to determine a personal foot model. The foot scan, as described above, may be provided, in part, from a smartphone, computer, tablet, scanning module, or any other scanning or imaging device. The method 2400 also typically includes conducting a physical post-wear analysis on the previously worn footwear as described above with reference to FIG. 23. In some configurations, the method 2400 includes determining 2420 an updated foot model based on the post-wear analysis described above with reference to FIG. 23, and the personal foot model of step 2410.

The method 2400 also includes generating or updating 2425 a design for a personalized footwear product (including a design for a personalized modular last and/or personalized shoe component, as disclosed herein), based at least in part on the personal foot model and/or the updated foot model. The updates to the design for the personalized footwear product may include, for example, updates to the selected personalized modular last (its selected modular core components, its selected last extensions and the locations of the extensions), and updates to the personalized shoe components (e.g., the type or number of components included, the dimensions of the components, the materials of the components). The method 2400 also includes outputting 2430 the design to a manufacturer or a manufacturing device.

The method 2400 also may include manufacturing 2435 the footwear product. Manufacturing the footwear product may be done through the use of additive, subtractive, and near net shape manufacturing and may include 3D printers, laser cutters, 3D knitting devices, a third-party manufacturer, or any other manufacturing means, and as described hereinabove.

Once the manufactured footwear has been worn for a duration of time, the user may return the footwear for analysis. Accordingly, in step 2440, the method includes receiving 2440 previously worn footwear. The duration of time may include, for example, about 6 months, however shorter and longer durations of time are contemplated herein. In practice, once the user has worn the shoes for a duration of time, the user may return the worn footwear such that the method includes receiving 2440 previously worn footwear. Further, the method 2400 may continue after receiving 2440 the footwear to (a) step 2410 described above regarding receiving and/or analyzing a foot scan and/or (b) step 2415 such that the method conducts additional post-wear analysis of the previously worn personalized footwear. In such a configuration, the user profile may be updated accordingly through the use of a processor or an electronic user device.

It is further contemplated that an automated user footwear last may be created from the updated personalized footwear last or customized user footwear last. The updated personalized footwear last or customized user footwear last may be further updated through the use of an algorithm which may be a machine learning algorithm to create the automated user footwear last.

In some embodiments, the system may include a footwear receiving and processing center. It is further contemplated that these centers may be separate facilities (such as a footwear receiving center and a footwear processing center). By means of this center (or centers) a previously worn footwear product worn by the user may be received, and a physical post-wear analysis may be conducted on the previously worn footwear product. The post-wear analysis feedback may then be obtained by the systems and methods herein to update the designs for the footwear product. The processing center may include a previously worn-footwear analysis module, a footwear deconstruction module, and a footwear recycling module, and may use methods similar to those described above with reference to FIG. 23. The post-wear analysis may be conducted physically by means of a human operator, or automatically, for example through the use of a scanning device.

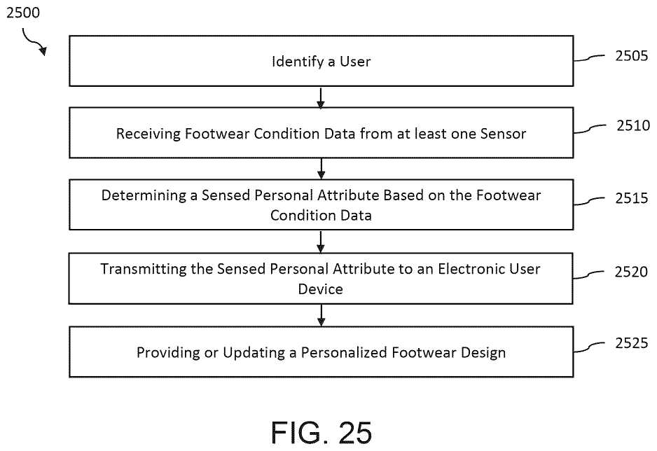

FIG. 25 illustrates an exemplary method 2500 of updating a footwear design in accordance with some embodiments. The method 2500 includes identifying 2505 a user. The method 2500 also includes a processor receiving 2510 footwear condition data from at least one sensor. The method 2500 also includes the processor determining 2515 a sensed personal attribute based on the footwear condition data. The method 2500 also includes the processor transmitting 2520 the sensed personal attribute to an electronic user device. The method 2500 also includes updating 2525 a personalized footwear design. The processor of method 2500 may be one or more processors, such as one in the footwear and/or a second processor in the electronic user device. The one or more processors or electronic user devices may communicate and transmit data directly or indirectly throughout the method or process.

In one illustrative embodiment of providing a personalized footwear recommendation, at least one electronic device, including an image sensor (e.g., a camera), at least one sensor, and at least one processor communicable with one another may be used to provide a personalized footwear recommendation. The at least one processor may receive image data from the electronic user device and/or the at least one sensor. The image data may include at least a portion of at least one foot of a user.

The at least one processor determines at least one foot shape attribute to associate with the user based, at least in part, on the image data. A user profile may be created and updated accordingly by at least one of the at least one processor or the at least one electronic user device. Determining the at least one foot shape attribute is similar to the analysis described in detail above with reference to FIG. 5.

The at least one processor also may determine at least one pathology attribute to associate with the user based, at least in part, on the image data. The user profile may be further updated accordingly by at least one of the at least one processor or the at least one electronic user device. Determining the at least pathology attribute is similar to the analysis described in detail above with reference to FIG. 7.

The at least one processor may also receive video data captured by the image sensor. The video data may include a video of the user's gait, such as the user walking. The at least one process may also determine at least one gait attribute associated with the user based, at least in part, on the video data. The user profile may be further updated accordingly by at least one of the at least one processor or the at least one electronic user device. Determining the at least gait attribute is similar to the analysis described in detail above with reference to FIG. 8.

The at least one processor may also receive information regarding a physical or contextual personal attribute. The at least one processor may receive this information via the user interface associated with the electronic user device. The physical or contextual personal attributes being those described in detail above, with reference to FIG. 3. The user profile may be further updated accordingly by at least one of the at least one processor or the at least one electronic user device. Determining the at least physical or contextual personal attribute is similar to the analysis described in detail above with reference to FIG. 10.

The at least one processor may also receive information regarding a post-wear analysis. The at least one processor may receive this information via the user interface associated with the electronic user device. The at least one processor may also determine a post-wear attribute to associate with the user. The user profile may be further updated accordingly by at least one of the at least one processor or the at least one electronic user device. Determining the post-wear attribute is similar to the analysis described in detail above with reference to FIG. 23.

The at least one processor may also receive information regarding footwear condition data. The at least one processor may receive this information via the at least one sensor. The at least one processor may also determine a sensed personal attribute to associate with the user. The user profile may be further updated accordingly by at least one of the at least one processor or the at least one electronic user device. Determining the sensed personal attribute is similar to the analysis described in detail above with reference to FIG. 21.

The at least one processor may also receive information regarding a microclimate analysis. The at least one processor may receive this information via the user interface associated with the electronic user device. The at least one processor may also determine a personal microclimate attribute to associate with the user. The user profile may be further updated accordingly by at least one of the at least one processor or the at least one electronic user device. Determining the personal microclimate attribute is similar to the analysis described in detail above with reference to FIG. 20.

The at least one processor may determine at least one footwear recommendation based on the above attributes to provide to the user via the user interface associated with the at least one electronic user device.

In some aspects, the techniques described herein relate to a system for providing a personalized footwear product, the system including at least one processor communicable with at least one electronic user device, the processor configured to obtain assessment information associated with a user, the assessment information including at least one foot measurement of the user, and generate a design for a personalized modular last, the personalized modular last including a modular core last. In some embodiments, generating the design for the personalized modular last includes determining a modular core last model by selecting one or more modular core last model components from a library of modular core last model components, based at least in part on the at least one foot measurement of the user.

In some embodiments, the processor is further configured to generate a design for at least one personalized shoe component based on the design for the personalized modular last and/or the assessment information associated with the user.

By some approaches, the processor is configured to send the design for the personalized modular last and the design for the at least one personalized shoe component to be manufactured. For example, the system may include a manufacturing execution system and the processor is configured to send the design for the personalized modular last and/or the design for the at least one personalized shoe component to the manufacturing execution system. In some embodiments, the manufacturing execution system is configured to manufacture the at least one personalized shoe component, provide the personalized modular last, use the personalized modular last to manufacture the personalized footwear product, and include the at least one personalized shoe component in the personalized footwear product.

In some aspects, generating the design for the personalized modular last further includes determining whether to include one or more last extensions in the personalized modular last based on the assessment information associated with the user. Generating the design for the personalized modular last further may also include determining one or more last extensions to be included in the personalized modular last based on the assessment information associated with the user.

In some embodiments, the personalized modular last includes a unary modular core last and the one or more last extensions removably coupled thereto, the unary modular core last including a single modular core last component and the processor is configured to determine the modular core last model by selecting one modular core last model component from the library of modular core last model components. In other approaches, the modular core last is a binary core last including two different modular core last components configured to be removably coupled together, for example, via at least one of interlocking pieces, mechanical fasteners, hook and loop fasteners, magnets, and removable adhesive. The processor may be configured to determine the modular core last model by selecting two different modular core last model components from the library of modular core last model components to permit further customization of the modular core last

In other approaches, the modular core last includes three or more different modular core last components configured to be removably coupled together, for example via at least one of interlocking pieces, mechanical fasteners, hook and loop fasteners, magnets, and removable adhesive, and the processor is configured to determine the modular core last model by selecting three or more different modular core last model components from the library of modular core last model components to permit further customization of the modular core last.

In some aspects, the assessment information associated with the user further includes foot shape information, pathology information, gait information, biomechanics information, and/or contextual information. Generating a design for the personalized modular last may be based at least in part on the foot shape information, pathology information, gait information, biomechanics information, and/or contextual information.

In some embodiments, the processor may be further configured to obtain feedback regarding a previously worn footwear product that was manufactured based on the design for the personalized modular last and the design for the personalized shoe component. In addition, the processor may be configured to update the design for the personalized modular last and/or update the design for the at least one personalized shoe component based at least in part on the feedback regarding a previously worn footwear product.

The system may also include that the assessment information associated with the user includes feedback regarding a previously worn footwear product, and generating the design for the personalized modular last and/or the design for the at least one shoe component is based at least in part on the feedback regarding a previously worn footwear product. By one approach, the feedback regarding a previously worn footwear product includes footwear condition data, including at least one of accelerometer sensor data, temperature sensor data, humidity sensor data, and pressure sensor data. In an embodiment, the feedback is obtained from a physical post-wear analysis of the previously worn footwear product.

In some aspects, the processor is configured to determine at least one sensed personal attribute based on the footwear condition data, and the design for the personalized modular last and/or the design for the at least one shoe component is based at least in part on the sensed personal attribute.

In a further approach, the system may include a footwear processing center, wherein the footwear processing center is operable to conduct a physical post-wear analysis on the previously worn footwear product, and wherein the feedback obtained by the processor is based on the physical post-wear analysis.

In some embodiments, the processor of the system is configured to determine one or more personal attributes associated with the user from the assessment information, wherein the one or more personal attributes include one or more of a personal foot shape attribute, a personal pathology attribute, a personal gait attribute, a personal biomechanics attribute, and a personal contextual attribute. In this approach, generating a design for the personalized modular last may be based at least in part on the one or more personal attributes.

In some embodiments, the assessment information includes image data, the image data including an image of at least a portion of at least one foot of the user, and the processor is configured to determine at least one personal foot shape attribute associated with the user by analyzing the image data to determine the at least one personal foot shape attribute. In this approach, generating a design for the personalized modular last may be based at least in part on the at least one personal foot shape attribute.

In some approaches, the processor may be configured to determine at least one personal pathology attribute associated with the user by analyzing the image data, and generating a design for the personalized modular last is based at least in part on the at least one personal pathology attribute.

In some aspects, the assessment information further includes video data such as video of a gait of the user, and the processor is configured to determine at least one personal gait attribute associated with the user by analyzing the video data. Generating a design for the personalized modular last may be based at least in part on the at least one personal gait attribute.

In some embodiments, the assessment information further includes sensor data, such as biomechanical data of the user, and the processor is configured to determine at least one personal biomechanics attribute associated with the user by analyzing the sensor data. Generating a design for the personalized modular last may be based at least in part on the at least one personal biomechanics attribute.

In some embodiments, the processor may be configured to determine the at least one personal pathology attribute using at least one machine learning algorithm trained to identify at least one observable foot pathology based on image recognition. The processor may be further configured to determine the at least one gait attribute using at least one machine learning algorithm trained to track points on a user based on a gait video of the user and to associate gait patterns or characteristics with the user based on the tracked points.

In some approaches, the processor may be configured to determine a modular core last model using at least one machine learning algorithm and/or determine the one or more last extensions using at least one machine learning algorithm. The processor may also be configured to use at least one machine learning algorithm to generate the design for the at least one personalized shoe component.

In some aspects, generating the design for the personalized modular last further includes determining one or more last extensions to be attached to the modular core last based on the assessment information associated with the user. By one approach, the manufacturing execution system is configured to manufacture the one or more last extensions and provide the personalized modular last by assembling the personalized modular last, wherein assembling the personalized modular last includes removably coupling the one or more last extensions to the modular core last based on the design for the personalized modular last. In some approaches, the removable coupling includes interlocking pieces, mechanical fasteners, hook and loop fasteners, magnets, and/or removable adhesive.

By one approach, the modular core last and the one or more last extensions are decouplable after use in manufacturing the personalized footwear, and re-usable to assemble one or more different personalized modular lasts to provide different personalized footwear products for the user or for one or more different individuals.

In some embodiments, the last extensions include primary last extensions and/or secondary last extensions. The last extensions may include one or more primary last extensions, wherein the one or more primary last extensions are removably couplable to at least one of an inner ball region, outer ball region, medial heel region, lateral heel region, toe tip region, ball girth region, instep region, or bottom region of the modular core last. In some approaches, the one or more last extensions include one or more secondary last extensions that are removably couplable to the modular core last at selected attachment points to correspond to a foot shape abnormality or a pathology of the user.

In one approach, the last extensions include one or more secondary last extensions, wherein at least one of the one or more secondary last extensions are removably coupled to at least one of the one or more primary last extensions.

In some embodiments, the design for the at least one personalized shoe component includes a shape of an upper, a shape of an insole, a shape of a midsole, a shape of an outsole, and/or an ancillary design element. The processor may be configured to generate the design for the at least one personalized shoe component using an algorithm to resize preset shoe components based on the design for the personalized modular last and/or the assessment information associated with the user. Generating the design for the at least one personalized shoe component may also include determining one or more materials for the at least one personalized shoe component based on the assessment information associated with the user.

In some approaches, the manufacturing execution system may be configured to manufacture the at least one personalized shoe component via laser cutting out of a sheet of material, 3D printing, and/or 3D knitting.

In some embodiments, the sheet of material for an upper is a natural leather or a fabric. In addition, the sheet of material for an insole may be a polyester foam, a polyethylene foam, a polyurethane foam, an EVA foam, or a cork. The sheet of material for a midsole may be open cell polyurethane, closed cell EVA, or Gore-Tex. In one approach, the sheet of material for an outsole is EVA or a rubber including no chlorine.

In some embodiments, assessment information is obtained from the electronic user device.

In some aspects, the techniques described herein relate to a modular last for personalized footwear, including: a modular core last and one or more last extensions, wherein the modular core last includes one or more modular core last components selected from a group of different modular core last components based at least in part on assessment information associated with a user, the assessment information including at least one foot measurement of the user. In some approaches, the one or more last extensions are removably coupled to the modular core last at selected attachment points on the modular core last, and the one or more last extensions and selected attachment points are selected based at least in part on the assessment information associated with the user.

In some approaches, the modular core last is a unary core last including a single modular core last component. In other approaches, the modular core last includes two or more different modular core last components configured to be removably coupled together, the two or more different modular core last components selected from the group of different modular core last components to permit further customization of the modular core last to the assessment information of the user. The two or more different modular core last components are interlocking components.

In one embodiment, the modular core last is a binary core last including two different modular core last components, for example, a core forefoot component and a core heel component. In another embodiment, the modular core last is a ternary core last including three different modular core last components, for example, a core forefoot component, a core midfoot component, and a core heel component. In a further embodiment, the modular core last is a quaternary core last including four different modular core last components, for example, a core toe tip component, a core ball component, a core midfoot component, and a core heel component.

In some aspects, the techniques described herein relate to a modular last, wherein the one or more modular core last components are selected via a machine learning algorithm which determines a close match to the assessment information associated with the user.

In some approaches, the modular last includes at least a first and a second last extension, wherein the first last extension is removably coupled to the modular core and the second last extension is removably coupled to the first last extension. By one approach, the second last extension is selected to correspond to a foot shape abnormality or a pathology of the user. In some embodiments, at least one of the one or more last extensions is removably coupled to a region of the modular core last via at least one of interlocking pieces, mechanical fasteners, hook and loop fasteners, magnets, and removable adhesive, wherein the region is an inner ball region, an outer ball region, a medial heel region, a lateral heel region, a ball girth region, a toe tip region, an instep region, or a bottom region.

In some aspects, the techniques described herein relate to a system for providing a personalized footwear product. The system may include at least one processor communicable with at least one electronic user device, the processor configured to obtain assessment information associated with a user, the assessment information including at least one foot measurement of the user; determine one or more footwear attributes associated with the user based on the assessment information; and generate a design for a personalized modular last, the personalized modular last including a modular core last. In some approaches, generating the design for the personalized modular last includes determining a modular core last model by selecting one or more modular core last model components from a library of modular core last model components, based at least in part on at least one of the one or more footwear attributes associated with the user. The processor may be further configured to generate a design for at least one personalized shoe component, based at least in part on the design for the personalized modular last, and, optionally, one or more of the one or more footwear attributes associated with the user; and wherein the processor is configured to send the design for the personalized modular last and the design for the at least one personalized shoe component to be manufactured.

In some embodiments, the system also includes a manufacturing execution system, wherein the processor is configured to send the design for the personalized modular last and the design for the at least one personalized shoe component to the manufacturing execution system. The manufacturing execution system may be configured to manufacture the at least one personalized shoe component, provide the personalized modular last, use the personalized modular last to manufacture the personalized footwear product, and include the at least one personalized shoe component in the personalized footwear product.

In some aspects, the techniques described herein relate to a system for providing a personalized modular last. The system may include at least one processor communicable with at least one electronic user device, the processor configured to obtain assessment information associated with a user, the assessment information including at least one foot measurement of the user, and generate a design for a personalized modular last that includes a modular core last. In some embodiments, generating the design for the personalized modular last includes determining a modular core last model by selecting one or more modular core last model components from a library of modular core last model components, based at least in part on the assessment information.

In some embodiments, the processor is configured to send the design for the personalized modular last to be manufactured. In one approach, the system includes a manufacturing execution system, and the processor is configured to send the design for the personalized modular last to the manufacturing execution system and the manufacturing execution system is configured to provide the personalized modular last.

In one approach, generating the design for the personalized modular last further includes determining whether to include one or more last extensions in the personalized modular last based on the assessment information associated with the user. Further, generating the design for the personalized modular last may include determining one or more last extensions to be included in the personalized modular last based on the assessment information associated with the user.

In one approach, the personalized modular last may include a unary modular core last and the one or more last extensions removably coupled thereto, wherein the unary modular core last includes a single modular core last component and the processor is configured to determine the modular core last model by selecting one modular core last model component from the library of modular core last model components. In other approaches, the modular core last may include two or more different modular core last components configured to be removably coupled together via at least one of interlocking pieces, mechanical fasteners, hook and loop fasteners, magnets, and removable adhesive, and the processor is configured to determine the modular core last model by selecting two or more different modular core last model components from the library of modular core last model components to permit further customization of the modular core last and provide a close match to the foot measurements the user.

In some aspects, the techniques described herein relate to a method, including obtaining assessment information associated with a user, the assessment information including at least one foot measurement of the user; and generating a design for a personalized modular last, wherein the personalized modular last includes a modular core last, wherein generating the design for the personalized modular last includes determining a modular core last model by selecting one or more modular core last model components from a library of modular core last model components, based at least in part on the at least one foot measurement of the user. The method may also include generating a design for at least one personalized shoe component, based at least in part on the design for the personalized modular last and, optionally, the assessment information associated with the user.

In some aspects, the method may include manufacturing a personalized footwear product based on the design for the personalized modular last and/or the design for the at least one personalized shoe component. Manufacturing a personalized footwear product may include providing the personalized modular last, using the personalized modular last to manufacture the personalized footwear product, and incorporating the at least one personalized shoe component into the personalized footwear product.

In some approaches the method may include obtaining feedback regarding a previously worn footwear product that was manufactured based on the design for the personalized modular last and the design for the personalized shoe component and updating the design for the personalized modular last and/or updating the design for the at least one personalized shoe component based at least in part on the feedback regarding a previously worn footwear product.

In some embodiments, the assessment information associated with the user includes feedback regarding a previously worn footwear product; and generating the design for the personalized modular last and/or generating the design for the at least one shoe component is based at least in part on the feedback regarding a previously worn footwear product. In some approaches, the feedback regarding the previously worn footwear product includes footwear condition data, including at least one of accelerometer sensor data, temperature sensor data, humidity sensor data, and pressure sensor data. By one approach, the feedback is obtained from a physical post-wear analysis of the previously worn footwear product.

The method may further include receiving a previously worn footwear product worn by the user and conducting a physical post-wear analysis on the previously worn footwear product, and wherein the feedback regarding the previously worn footwear product is based on the physical post-wear analysis.

In some approaches the assessment information associated with the user further includes foot shape information, pathology information, gait information, biomechanics information, and/or contextual information. In these approaches, generating a design for the personalized modular last may be based at least in part on the foot shape information, pathology information, gait information, biomechanics information, and/or contextual information.

The method may further provide that generating the design for the personalized modular last includes determining one or more last extensions to be included in the personalized modular last based on the assessment information associated with the user.

In some approaches, the method may provide that the personalized modular last includes a unary modular core last and the one or more last extensions are removably coupled thereto, wherein the unary modular core last includes a single modular core last component and the modular core last model is determined by selecting one modular core last model component from the library of modular core last model components.

In other approaches, the method may provide that the personalized modular core last includes two or more different modular core last components configured to be removably coupled together via at least one of interlocking pieces, mechanical fasteners, hook and loop fasteners, magnets, and removable adhesive, and the modular core last model is determined by selecting two or more different modular core last model components from the library of modular core last model components to permit further customization of the modular core last to the assessment information associated with the user.

In some aspects, the techniques described herein relate to a system for providing a personalized footwear product, the system including at least one processor communicable with at least one electronic user device and the processor configured to obtain assessment information associated with a user, the assessment information including feedback regarding a previously worn footwear product, and generate an updated design for a personalized footwear product. In some embodiments, the updated design for a personalized footwear product includes a design for a personalized modular last and a design for at least one personalized shoe component, wherein the design for the personalized modular last includes a modular core last model which is determined by selecting one or more modular core last model components from a library of modular core last model components, based at least in part on the feedback regarding a previously worn footwear product. The design for the at least one personalized shoe component may be based at least in part on the design for the personalized modular last and/or the feedback regarding a previously worn footwear product.

In some embodiments, the feedback regarding a previously worn footwear product includes at least one of: foot shape information, gait information, biomechanics information, pathology information, and contextual information. In one approach, the feedback regarding a previously worn footwear product includes footwear condition data, including at least one of accelerometer sensor data, temperature sensor data, humidity sensor data, and pressure sensor data.

In some aspects, the techniques described herein relate to a system, wherein the processor is configured to determine at least one sensed personal attribute based on the footwear condition data, and the updated design for the personalized footwear product is based at least in part on the sense personal attribute. By one approach, the footwear condition data includes accelerometer sensor data, and the processor is configured to analyze the accelerometer sensor data to determine foot alignment. In another approach, the footwear condition data includes temperature sensor data, and the processor is configured to analyze the temperature sensor data to determine an internal temperature of one or more regions of the footwear product. In some embodiments, the footwear condition data includes humidity sensor data, and the processor is configured to analyze the humidity sensor data to determine a moisture level of the footwear product. In addition, the footwear condition data may include pressure sensor data, and the processor may be configured to analyze the pressure data to determine a pressure distribution on the sole of the footwear product. The footwear condition data may include at least one of a humidity sensor and a temperature sensor, and the processor may be configured to determine a microclimate of the footwear product.

In one approach, the feedback is obtained from a physical post-wear analysis of the previously worn footwear product.

In some embodiments, the system includes a footwear processing center, wherein the footwear processing center is operable to conduct a physical post-wear analysis on the previously worn footwear product, and wherein the feedback obtained by the processor is based on the physical post-wear analysis. In some approaches, the feedback regarding a previously worn footwear product is provided by the user.

In embodiments, the processor is configured to generate the updated design for the personalized footwear product by using the feedback to determine the design for the personalized modular last. The updated design for the personalized footwear product may include at least one change of construction, at least one change of material, or a combination of least one change of construction and at least one change of material for the personalized footwear product compared to the previously worn footwear product.

In some configurations, the processor is configured to generate the updated design for the personalized footwear product by using the feedback to determine one or more last extensions to be added to the design of the personalized modular last. The system may also include a user profile, the processor being further configured to update a user profile based on the feedback regarding the previously worn footwear product.

In some aspects, the techniques described herein relate to a system for providing a personalized footwear product, the system including at least one processor communicable with at least one electronic user device and the processor configured to obtain assessment information associated with a user, the assessment information including at least one foot measurement of the user, and generate a design for a personalized modular last, wherein the personalized modular last includes a modular core last. In some approaches, generating the design for the personalized modular last includes determining a modular core last model by selecting one or more modular core last model components from a library of modular core last model components, based at least in part on the at least one foot measurement of the user. The processor may further be configured to generate a design for at least one personalized shoe component, based on the design for the personalized modular last and, optionally, the assessment information associated with the user, In addition, the processor may also be configured to obtain feedback regarding a previously worn footwear product that was manufactured based on the design for the personalized modular last and the design for the personalized shoe component, and the processor may generate an updated design for the personalized modular last and/or an updated design for the at least one personalized shoe component based at least in part on the feedback regarding a previously worn footwear product. The feedback, for example, may be feedback is obtained from a physical post-wear analysis of the previously worn footwear product. In one approach, the system includes a footwear processing center operable to conduct a physical post-wear analysis on the previously worn footwear product, and wherein the feedback obtained by the processor is based on the physical post-wear analysis.

In some approaches, the feedback regarding a previously worn footwear product includes footwear condition data, including at least one of accelerometer sensor data, temperature sensor data, humidity sensor data, and pressure sensor data.

In addition, the processor may be configured to determine at least one sensed personal attribute based on the footwear condition data, and the updated design for the personalized footwear product is based at least in part on the sense personal attribute.

In some embodiments, the techniques described herein relate to a method for designing one or more personalized shoe components, including obtaining assessment information associated with a user, the assessment information including at least one foot measurement of the user; and generating a design for one or more personalized shoe components. In some embodiments, generating the design for the one or more personalized shoe components includes, for each of the one or more personalized shoe components, using an algorithm to select a preset shoe component design from a library of preset shoe component designs based on a design for a personalized modular last and, optionally, the assessment information associated with the user; optionally, using an algorithm to resize the selected preset shoe component design based on the design for the personalized modular last and, optionally, the assessment information associated with the user; and selecting a material for the personalized shoe component based on the design for the personalized modular last and/or the assessment information associated with the user.

In one approach, the one or more personalized shoe components include at least one of an upper component, an insole component, a midsole component, and an outsole component. In another approach, the one or more personalized shoe components include at least two of an upper component, an insole component, a midsole component, and an outsole component. In a further approach, the one or more personalized shoe components include at least three of an upper component, an insole component, a midsole component, and an outsole component. In an additional approach, the one or more personalized shoe components include an upper component, an insole component, a midsole component, and an outsole component.

The method may also provide that generating the design for the one or more personalized shoe components further includes determining at least one ancillary design element to be added to the design. In embodiments, the at least one ancillary design element includes one or more of laces, straps, air bubbles, lights, traction materials, extra padding, outsole thread pattern, specific textures, foxing, eyestays, eyelets, seams, stitching, collar padding, toe tip reinforcement, added toe spring, vamp lining, printed and/or embossed designs, and extra panels.

In some embodiments, the techniques described herein relate to a method for designing a personalized shoe component including obtaining assessment information associated with a user, the assessment information including at least one foot measurement of the user, and generating a design for a personalized upper component. In embodiments, generating the design for the personalized upper component includes using an algorithm to select a preset upper design from a library of preset upper designs based on a design for a personalized modular last personalized to the user and, optionally, the assessment information associated with the user; optionally, using an algorithm to resize the selected preset upper design based on the design for the personalized modular last personalized to the user and, optionally, the assessment information associated with the user; and selecting a material for the personalized upper component based on the design for the personalized modular last personalized to the user and/or the assessment information associated with the user.

In some aspects, the techniques described herein relate to a method for designing a personalized shoe component, including obtaining assessment information associated with a user, the assessment information including at least one foot measurement of the user, and generating a design for a personalized insole component. Generating the design for the personalized insole component may include using an algorithm to select a preset insole design from a library of insole upper designs based on a design for a personalized modular last personalized to the user and, optionally, the assessment information associated with the user; optionally, using an algorithm to resize the selected preset insole design based on the design for the personalized modular last personalized to the user and, optionally, the assessment information associated with the user; and selecting a material for the personalized insole component based on the design for the personalized modular last personalized to the user and/or the assessment information associated with the user.

In some aspects, the techniques described herein relate to a method for designing a personalized shoe component, including obtaining assessment information associated with a user, the assessment information including at least one foot measurement of the user, and generating a design for a personalized midsole component. In embodiments, generating the design for the personalized midsole component includes: using an algorithm to select a preset upper design from a library of preset midsole designs based on a design for a personalized modular last personalized to the user and, optionally, the assessment information associated with the user; optionally, using an algorithm to resize the selected preset midsole design based on the design for the personalized modular last and, optionally, the assessment information associated with the user; and selecting a material for the personalized midsole component based on the design for the personalized modular last and/or the assessment information associated with the user.

In some aspects, the techniques described herein relate to a method for designing a personalized shoe component, including obtaining assessment information associated with a user, the assessment information including at least one foot measurement of the user, and generating a design for a personalized outsole component. In embodiments, generating the design for the personalized outsole component includes: using an algorithm to select a preset upper design from a library of preset outsole designs based on a design for a personalized modular last personalized to the user and, optionally, the assessment information associated with the user; optionally, using an algorithm to resize the selected preset outsole design based on the design for the personalized modular last and, optionally, the assessment information associated with the user; and selecting a material for the personalized outsole component based on the design for the personalized modular last and/or the assessment information associated with the user.

In some aspects, the techniques described herein relate to a method for designing one or more personalized shoe components, including obtaining assessment information associated with a user, the assessment information including at least one foot measurement of the user, and generating a design for a personalized outsole component. In embodiments, generating the design for the personalized outsole component includes: using an algorithm to select a preset upper design from a library of preset outsole designs based on a design for a personalized modular last personalized to the user and, optionally, the assessment information associated with the user; optionally, using an algorithm to resize the selected preset outsole design based on the design for the personalized modular last and, optionally, the assessment information associated with the user; and selecting a material for the personalized outsole component based on the design for the personalized modular last and/or the assessment information associated with the user.

Those skilled in the art will recognize that a wide variety of other modifications, alterations, and combinations can also be made with respect to the above-described embodiments without departing from the scope of the disclosure, and that such modifications, alterations, and combinations are to be viewed as being within the ambit of the inventive concept.

1. A system for personalizing footwear, comprising:
at least one processor communicable with at least one electronic user device, the processor configured to:
obtain assessment information associated with a user, the assessment information comprising at least one foot measurement of the user, and
generate a design for a personalized modular last, wherein the personalized modular last comprises a modular core last,
wherein generating the design for the personalized modular last includes determining a modular core last model by selecting one or more modular core last model components from a library of modular core last model components, based at least in part on the at least one foot measurement of the user;
the processor further configured to generate a design for at least one personalized shoe component, based on the design for the personalized modular last and/or the assessment information associated with the user.

2. The system of claim 1, further comprising a manufacturing execution system, wherein the processor is configured to send the design for the personalized modular last and the design for the at least one personalized shoe component to the manufacturing execution system for manufacturing, and the manufacturing execution system is configured to manufacture the at least one personalized shoe component, provide the personalized modular last, use the personalized modular last to manufacture a personalized footwear product, and include the at least one personalized shoe component in the personalized footwear product.

3. (canceled)

4. The system of claim 1, wherein generating the design for the personalized modular last further includes determining one or more last extensions to be coupled to the personalized modular last based on the assessment information associated with the user.

5. The system of claim 4, wherein the personalized modular last comprises a unary modular core last and the one or more last extensions coupled thereto, wherein the unary modular core last comprises a single modular core last component and the processor is configured to determine the modular core last model by selecting one modular core last model component from the library of modular core last model components.

6. The system of claim 1, wherein the modular core last comprises two or more different modular core last components configured to be removably coupled together, and the processor is configured to determine the modular core last model by selecting two or more different modular core last model components from the library of modular core last model components to permit further customization of the modular core last and provide a close match to the foot measurements the user.

7. The system of claim 1, wherein the assessment information associated with the user further comprises foot shape information, pathology information, gait information, biomechanics information, and/or contextual information; and
wherein generating a design for the personalized modular last is based at least in part on the foot shape information, pathology information, gait information, biomechanics information, and/or contextual information.

8. The system of claim 1, wherein the assessment information includes image data, the image data including an image of at least a portion of at least one foot of the user, and the processor is configured to determine at least one personal pathology attribute associated with the user by analyzing the image data;
and wherein generating a design for the personalized modular last is based at least in part on the at least one personal pathology attribute.

9. The system of claim 8, wherein the processor is configured to determine the at least one personal pathology attribute using at least one machine learning algorithm trained to identify at least one observable foot pathology based on image recognition.

10. The system of claim 1, wherein the processor is further configured to obtain feedback regarding a previously worn footwear product that was manufactured based on the design for the personalized modular last and the design for the personalized shoe component; and
the processor is further configured to generate an updated design for the personalized modular last and/or an updated design for the at least one personalized shoe component based at least in part on the feedback regarding a previously worn footwear product.

11. (canceled)

12. The system of claim 1, wherein the personalized shoe component includes a shape of an upper, a shape of an insole, a shape of a midsole, a shape of an outsole, and/or an ancillary design element.

13. (canceled)

14. (canceled)

15. A modular last for personalized footwear, comprising:
a modular core last and one or more last extensions,
wherein the modular core last comprises one or more modular core last components selected from a group of different modular core last components based at least in part on assessment information associated with a user, the assessment information comprising at least one foot measurement of the user,
wherein the one or more last extensions are removably coupled to the modular core last at selected attachment points on the modular core last, the one or more last extensions and selected attachment points selected based at least in part on the assessment information associated with the user.

16. The modular last of claim 15, wherein the modular core last comprises two or more different modular core last components configured to be removably coupled together, the two or more different modular core last components selected from the group of different modular core last components to permit further customization of the modular core last to the assessment information of the user.

17. The modular last of claim 16, wherein the modular core last is a binary core last, a ternary core last, or a quaternary core last, the binary core last comprising a core forefoot component and a core heel component, the ternary core last comprising a core forefoot component, a core midfoot component, and a core heel component, and the quaternary core last comprising a core toe tip component, a core ball component, a core midfoot component, and a core heel component.

18. The modular last of claim 15, wherein the one or more modular core last components are selected via a machine learning algorithm which determines a close match to the assessment information associated with the user

19. The modular last of claim 15, comprising at least a first and a second last extension, wherein the first last extension is removably coupled to the modular core last and the second last extension is removably coupled to the first last extension.

20. The modular last of claim 15, wherein at least one of the one or more last extensions is removably coupled to a region of the modular core last via at least one of interlocking pieces, mechanical fasteners, hook and loop fasteners, magnets, and removable adhesive, wherein the region is an inner ball region, an outer ball region, a medial heel region, a lateral heel region, a ball girth region, a toe tip region, an instep region, or a bottom region.

21. A method, comprising:
obtaining assessment information associated with a user, the assessment information comprising at least one foot measurement of the user;
generating a design for a personalized modular last, wherein the personalized modular last comprises a modular core last, wherein generating the design for the personalized modular last includes determining a modular core last model by selecting one or more modular core last model components from a library of modular core last model components, based at least in part on the at least one foot measurement of the user; and
generating a design for at least one personalized shoe component, based at least in part on the design for the personalized modular last and, optionally, the assessment information associated with the user.

22. The method of claim 21, further comprising manufacturing a personalized footwear product based on the design for the personalized modular last and the design for the at least one personalized shoe component, wherein the step of manufacturing a personalized footwear product includes providing the personalized modular last, using the personalized modular last to manufacture the personalized footwear product, and incorporating the at least one personalized shoe component into the personalized footwear product.

23. The method of claim 21, further comprising obtaining feedback regarding a previously worn footwear product that was manufactured based on the design for the personalized modular last and the design for the personalized shoe component; and
updating the design for the personalized modular last and/or updating the design for the at least one personalized shoe component based at least in part on the feedback regarding a previously worn footwear product.

24. The method of claim 21, wherein generating a design for one or more personalized shoe components includes, for each of the one or more personalized shoe components:
using an algorithm to select a preset shoe component design from a library of preset shoe component designs based on the design for a personalized modular last and, optionally, the assessment information associated with the user; and
optionally, using an algorithm to resize the selected preset shoe component design based on the design for the personalized modular last and, optionally, the assessment information associated with the user; and
selecting a material for the personalized shoe component based on the design for the personalized modular last and/or the assessment information associated with the user.
